(19) United States
(12) Patent Application Publication
(10) Pub. No.: US 2011/0130346 A1
(43) Pub. Date: Jun. 2, 2011

(54) PEPTIDE CONJUGATES FOR DELVERY OF BIOLOGICALLY ACTIVE COMPOUNDS

(75) Inventors: Matthew Wood, Oxford (GB); Haifang Yin, Oxford (GB)

(73) Assignee: ISIS INNOVATION LIMITED, Oxford (GB)

(21) Appl. No.: 12/995,380

(22) PCT Filed: Jun. 1, 2009
(86) PCT No.: PCT/GB09/01370

§ 371 Date: Feb. 15, 2011

Related U.S. Application Data
(63) Continuation-in-part of application No. 61/057,351, filed on May 30, 2008.

(51) Int. Cl. A61K 38/14; C07K 7/08; A61K 38/10; C07K 7/06; A61K 38/08; C07K 14/075; A61K 38/16; C07K 9/00; C12N 5/00; A61P 21/00

(52) U.S. Cl. 514/20.9; 530/327; 514/21.5; 530/329; 514/21.7; 530/326; 514/21.4; 530/322; 435/375

(57) ABSTRACT

A construct comprising a cell delivery peptide covalently attached to a biologically active compound suitable for delivery of said biologically active compound into cells, wherein optionally the cells are cardiac muscle, skeletal muscle, smooth muscle or contractile cells.

### The PRIORITY APPLICATION

The contents of the priority application (U.S. Application No. 61/057,351 filed 30 May 2008), including the sequences in that application, are incorporated herein by reference.

### FIELD OF THE INVENTION

The present invention relates to delivering molecules into a cell.

### BACKGROUND OF THE INVENTION

There is a need in the art for improved methods of facilitating uptake of compounds into cells, particularly to deliver therapeutic compounds to cells.

### SUMMARY OF THE INVENTION

The invention is based on characterisation of properties of substances that could facilitate delivery of compounds into cells.

Accordingly the invention provides a construct comprising a cell delivery peptide covalently or non-covalently attached to a biologically active compound suitable for delivery of said biologically active compound into cells, wherein the cell delivery peptide is selected from MSP, AAV6, AAV8, TAT or (RXR)₄; or a functional derivative thereof, and wherein optionally the cells are cardiac muscle, skeletal muscle, smooth muscle or contractile cells and optionally the MSP peptide is ASSLNIA, the AAV6 peptide is TVAVNLQSSSTDPATGDVHVM, the AAV8 peptide is IVADNLQQQNTAPQIGTVNSQ, the TAT peptide is YGRKKRRQRRRP or the (RXR)₄ peptide is RXRRXRRXRRXR wherein R is L-arginine and X is 6-aminohexanoic acid.

The inventors have also found that glucose analogues may be used to enhance uptake of molecules into cells.

### BRIEF DESCRIPTION OF THE FIGURES

FIG. 1 shows the evaluation of various peptide conjugates to neutral PNA in H2K mdx cells. A. Cell viability in H2K mdx cells following transfection with 2.5 or 25 μM PNA-peptide conjugates. B. RT-PCR analysis of exon 23 skipping (688 bp) following transfection with 250 nM of various PNA-peptide conjugates.

FIG. 2 illustrates Dystrophin expression following injection of various PNA-peptide conjugates. A. Immunofluorescent dystrophin staining in TA muscles of mdx mice of different ages, two weeks following injection of 5 μg of PNA-peptide conjugates (scale bar=200 μm). B. Quantitative evaluation of total dystrophin-positive fibres in TA muscles two weeks after a single intramuscular injection of PNA-peptide conjugates. All groups showed significant improvement in comparison with age-matched mdx mice (*P<0.05). C. Quantitative evaluation of total dystrophin-positive fibres in TA muscles of 2-month old mdx mice with increasing doses of PNA-peptide conjugates. AAV6 conjugate showed significant difference between 20 μg and 5, 10 μg injection (*P<0.05); AAV8 showed significant difference between 10 μg and 5 μg injection (*P<0.05).

FIG. 3 is a western blot analysis. Total protein was extracted from TA muscles of 2-month old mdx mice two weeks after a single intramuscular injection with 5 μg PNA-peptide conjugate. No visible difference in the size of dystrophins between muscle treated with PNA and muscle from the normal C57BL6 mouse.

FIG. 4 shows restoration of dystrophin expression in aged mdx mice. Restoration of dystrophin expression in aged mdx mice following single 5 ug intramuscular injections of PNA AOs in 12 month old mdx mice. (A).

FIGS. 5 and 6 show long-term correction of dystrophin expression following intramuscular PNA administration in mdx mice. (A) Immunohistochemistry to detect (scale bar=200 μm).

FIG. 7 shows systemic delivery of PNA AOs for dystrophin splice correction. 20 mer PNA AOs were administered by tail vein intravenous infusion to 6 week old mdx mice and the effects analysed 3 weeks post-injection. (A) (P<0.05).

FIGS. 8 to 10 show effects of PNA-peptide conjugates following intramuscular delivery in mdx mice.

FIGS. 11 to 13 show work concerning enhancement of delivering to cells using glucose analogues.

### DESCRIPTION OF SEQUENCES

The priority application shows the sequence of the human dystrophin gene and illustrates the location of the exons and introns. The sequence was obtained from the following web link:
http://vega.sanger.ac.uk/Homo_{—}sapiens/transview?transcript=OTTHUMT00000056182

The priority application also provides the partial sequence of the mouse dystrophin gene. The sequence illustrates the location of the exons and introns but does not show the full intron sequences. The full sequences can be accessed at the following web link:
http://vega.sanger.ac.uk/Mus_{—}musculus/transview?transcript=OTTMUST00000043357

SEQ ID NO: 1 is a PNA sequence of the invention.

SEQ ID NO: 2 to 124 are exon/intron boundary sequences that can be targeted by antisense oligonucleotide sequences.

SEQ ID NO: 125 to 128 are RT-PCR primer sequences.

### DETAILED DESCRIPTION OF THE INVENTION

### Peptide-Mediated Cell Delivery

Peptide-mediated cell delivery is the use of a peptide, either as noncovalent complexes or as covalent conjugates, to enhance the delivery of molecules, such as a biologically active compound, into cells. A peptide capable of effecting peptide-mediated cell delivery may be referred to as a “cell delivery peptide” or a “cell penetrating peptide”. The cell delivery peptide may be a tissue-specific peptide (such as MSP) or a transduction peptide (such as HIV TAT protein).

The invention also concerns use of glucose analogues to enhance delivery of molecules to cells, both in vitro and in vivo. This aspect of the invention may be used to deliver any of the nucleic acids or conjugates described herein, for example in diagnosis or therapy as described for any embodiment herein.

The inventors have discovered novel constructs comprising a cell delivery peptide linked to a biologically active compound suitable for delivery of said biologically active compound into cells, such as cardiac and skeletal muscle cells. These constructs can be used to deliver a biologically active compound into a cell in vivo or in vitro, and may be used in a method of treatment or diagnosis of the human or animal body. In particular, the constructs deliver a biologically active compound to cardiac and heart muscle cells, and therefore the constructs may be used in a method of treatment or diagnosis of a cardiac or skeletal muscle disease.

Different peptides were conjugated to PNA antisense oligonucleotides (AOs). Firstly, HIV TAT (referred to as TAT) is a well-studied 12 mer peptide that has been previously tested for delivering a range of different oligonucleotides in vitro and in vivo. Muscle-specific protein (MSP) is a 7 mer muscle-specific peptide, originally identified by screening a phage library in the mouse cell line C2C12, and here evaluated as a potential delivery peptide for the first time. AAV6 is a 21 mer peptide derived from a putative heparin-binding domain on the surface loop of the AAV6 capsid protein VP1 (576-597). AAV6 is reported to transfect skeletal muscle with high efficiency but its detailed structure is still unavailable. The AAV6 capsid protein VP1 was therefore compared with the well-characterised AAV2 capsid protein VP1 which identified the putative heparin-binding domain for cell tropism by bioinformatic analysis of AAV serotypes 1, 2, 6, 7 and 8 (data not shown). Another 21 mer peptide (578-599) from the AAV8 capsid protein VP1 was also identified through the same bioinformatic analysis. AAV8 has been reported to be highly effective at transfecting skeletal and cardiac muscle. The structures of the peptides are given in Table 3, where they are covalently linked to PNA sequences via a disulphide bridge and an AEEA linker. The peptides of TAT, MSP, AAV6 and AAV8 can be covalently linked with any biologically active compound to form the construct of the invention.

Preferably, the cell delivery peptide is attached to the biologically active compound by means of a disulphide bridge and an AEEA (2 aminoethoxy-2-ethoxy acetic acid) linker as illustrated in Table 3. The attachment may be by means of an amide linker (preferably a stable amide linker).

It will be understood that functional derivatives of the specific cell delivery peptides disclosed herein could be used. Such derivatives are typically peptides that have sequences which have homology to the original peptides. The derivatives may represent fragments of the original peptides or homologues, or may represent peptides that include insertions (amino acid additions) to the original peptides, homologues or said fragments. Typically the derivative has at least 70%, 80% or 90% of the number of amino acids present in the original peptide or may have less than 200% or 150% of the number of amino acids present in the original peptide. The derivative is generally able to enhance the delivery of a compound to a cell, for example as determined by any assay mentioned herein.

A biologically active compound comprised within the constructs of the invention is any compound that may exert a biological effect within a biological cell, typically affecting the expression of one or more genes in the cell. Examples of biologically active compounds include nucleic acids, peptides, proteins, DNAzymes, Ribozymes, chromophores, fluorophores and pharmaceuticals.

Such nucleic acids may be single or double stranded. Single-stranded nucleic acids include those with phosphodiester, 2′O-methyl, 2′ methoxy-ethyl, phosphoramidate, methylphosphonate, and/or phosphorothioate backbone chemistry, peptide nucleic acid (PNA), phosphorodiamidate morpholino oligonucleotide (PMO), locked nucleic acid (LNA), glycol nucleic acid (GNA) and threose nucleic acid (TNA). Double-stranded nucleic acids include plasmid DNA and small interfering RNAs (siRNAs).

The biologically active compound to be delivered is chosen on the basis of the desired effect of that compound on the cell into which it is delivered and the mechanism by which that effect is to be carried out. For example, the compound may be used to treat a disease state within that cell, for example by attenuating the propagation of a pathogen (e.g. a virus), typically by using a small-molecule inhibitor, or by correcting the expression of an aberrantly expressed protein, typically using an anti-sense oligonucleotide (AO) to modulate pre-mRNA splicing (see below). The compound may also be used to diagnose a disease state within that cell, for example by delivering to that cell a compound used to detect a diagnostic marker.

The skeletal muscle disease to be treated may be a muscular dystrophy phenotype, optionally Duchenne muscular dystrophy (DMD), Becker muscular dystrophy, myotonic dystrophy (MD), spinal muscular atrophy, limb-girdle muscular dystrophy (LGMD), facioscapulohumeral muscular dystrophy, congenital muscular dystrophy, oculpharyngeal muscular dystrophy (OMD), distal muscular dystrophy and Emery-Dreifuss muscular dystrophy (EDMD).

Genes implicated in the pathogenesis of these diseases include dystrophin (Duchenne muscular dystrophy and Becker muscular dystrophy), DMPK (DM1 type MD), ZNF9 (DM2 type MD), PABPN1 (OMD), emerin, lamin A or lamin C (EDMD), myotilin (LGMD-1A), lamin A/C (LGMD-1B), caveolin-3 (LGMD-1C), calpain-3 (LGMD-2A), dysferlin (LGMD-2B and Miyoshi myopathy), gamma-sarcoglycan LGMD-2C), alpha-sarcoglycan (LGMD-2D), betaa-sarcoglycan (LGMD-2E), delta-sarcoglycan (LGMD-2F and CMD1L), telethonin (LGMD-2G), TRIM32 (LGMD-2H), fukutin-related protein (LGMD-2I), titin (LGMD-2J), and O-mannosyltransferase-1 (LGMD-2K).

The cardiac muscle disease to be treated may be coronary heart disease, congenital heart disease, ischemic, hypertensive, inflammatory or intrinsic cardiomyopathy. Intrinsic cardiomyopathy includes the following disorders (with associated genes): dilated cardiomyopathy (dystrophin, G4.5, actin, desmin, delta-sarcoglycan, troponin T, beta-myosin heavy chain, alpha-tropomyosin, mitochondrial respiratory chain), dilated cardiomyopathy with conduction disease (lamin A/C), hypertrophic cardiomyopathy (beta-myosin heavy chain, troponin T, troponin I, alpha-tropomyosin, myosin-binding protein C, myosin essential light chain, myosin regulatory light chain, titin), hypertrophic cardiomyopathy with Wolff-Parkinson-White syndrome (AMPK, mitochondrial respiratory chain), and left ventricular noncompaction (G4.5, alpha-dystrobrevin).

In one embodiment the biologically active compound is not RNA. In another embodiment the biologically active compound is not siRNA. In one embodiment the cell delivery peptide is not TAT peptide.

### Modulation of Pre-RNA Splicing

DNA sequences are transcribed into pre-mRNAs which contain coding regions (exons) and generally also contain intervening non-coding regions (introns). Introns are removed from pre-mRNAs in a precise process called cis-splicing. Splicing takes place as a coordinated interaction of several small nuclear ribonucleoprotein particles (snRNPs) and many protein factors that assemble to form an enzymatic complex known as the spliceosome. Specific motifs in the pre-mRNA that are involved in the splicing process include splice site acceptors, splice site donors, exonic splicing enhancers (ESEs) and exon splicing silencers.

Pre-mRNA can be subject to various splicing events. Alternative splicing can result in several different mRNAs being capable of being produced from the same pre-mRNA. Alternative splicing can also occur through a mutation in the pre-mRNA, for instance generating an additional splice acceptor and/or splice donor sequence (cryptic sequences). Restructuring the exons in the pre-mRNA, by inducing exon skipping or inclusion, represents a means of correcting the expression from pre-mRNA exhibiting undesirable splicing or expression in an individual. Exon restructuring can be used to promote the production of a functional protein in a cell. Restructuring can lead to the generation of a coding region for a functional protein. This can be used to restore an open reading frame that was lost as a result of a mutation.

Antisense oligonucleotides (AOs) can be used to alter pre-mRNA processing via the targeted blockage of motifs involved in splicing. Hybridisation of antisense oligonucletides to splice site motifs prevents normal spliceosome assembly and results in the failure of the splicing machinery to recognize and include the target exon(s) in the mature gene transcript. This approach can be applied to diseases caused by aberrant splicing, or where alteration of normal splicing would abrogate the disease-causing mutation. This includes: (i) blockage of cryptic splice sites, (ii) exon removal or inclusion to alter isoform expression, and (iii) removal of exons to either eliminate a nonsense mutation or restore the reading frame around a genomic deletion.

An example of a gene in which the reading frame may be restored is the Duchenne muscular dystrophy (DMD) gene. The dystrophin protein is encoded by a plurality of exons over a range of at least 2.6 Mb. DMD is mainly caused by nonsense and frame-shift mutations in the dystrophin gene resulting in a deficiency in the expression of dystrophin protein. The dystrophin protein consists of two essential functional domains connected by a central rod domain. Dystrophin links the cytoskeleton to the extracellular matrix and is thought to be required to maintain muscle fibre stability during contraction. Mutations that disrupt the open reading frame result in prematurely truncated proteins unable to fulfill their bridge function. Ultimately this leads to muscle fibre damage and the continuous loss of muscle fibres, replacement of muscle tissue by fat and fibrotic tissue, impaired muscle function, and eventually the severe phenotype observed for DMD patients. In contrast, mutations that maintain the open reading frame allow for the generation of internally deleted, but partially functional, dystrophins. These mutations are associated with Becker muscular dystrophy (BMD), a much milder disease when compared with DMD. Patients generally remain ambulant until later in life and have near normal life expectancies.

The inventors have discovered that AOs based on peptide nucleic acid (PNAs) that are capable of targeting splice site motifs in mutated dystrophin mRNA can efficiently induce exon skipping. It is possible to target an exon which flanks an out-of frame deletion or duplication so that the reading frame can be restored and dystrophin production allowed. The removal of the mutated exon in this way allows shortened but functional (BMD-like) amounts of dystrophin protein to be produced. As a result, a severe DMD phenotype can be converted into a milder BMD phenotype.

Dystrophia myotonica (myotonic dystrophy) type 1 (DM1), the most common muscular dystrophy affecting adults, is caused by expansion of a CTG repeat in the 3′ untranslated region of the gene encoding the DM protein kinase (DMPK). Evidence suggests that DM1 is not caused by abnormal expression of DMPK protein, but rather that it involves a toxic gain of function by mutant DMPK transcripts that contain an expanded CUG repeat (CUG^{exp}). The transcripts containing a CUG^{exp} tract elicit abnormal regulation of alternative splicing, or spliceopathy. The splicing defect, which selectively affects a specific group of pre-mRNAs, is thought to result from reduced activity of splicing factors in the muscleblind (MBNL) family, increased levels of CUG-binding protein 1, or both. Myotonia in mouse models of DM appears to result from abnormal inclusion of exon 7a in the ClC-1 mRNA. Inclusion of exon 7a causes frame shift and introduction of a premature termination codon in the ClC-1 mRNA. A therapeutic strategy for myotonic dystrophy is therefore to repress the inclusion of exon 7a in the mouse ClC-1 mRNA, or the corresponding exon in human ClC-1 mRNA.

Just as targeted blockage of consensus splice sites and ESEs promotes exon exclusion, the blockage of exonic or intronic splicing silencers, or the introduction of splicing enhancer sequences, can enhance exon inclusion. This offers the ability to enhance expression of alternatively spliced ‘weak’ exons to induce the most functionally preferable isoform. In spinal muscular atrophy (SMA), mutations in the survival motor neuron (SMN1) gene are responsible for a degenerative disease that presents as childhood muscle weakness and, in the more serious forms, can cause fatal respiratory failure. The severity of the disease is modified by the production of SMN protein encoded by the paralogous gene, SMN2. Although SMN2 is nearly identical to SMN1, a silent C to T mutation in exon 7 abrogates an ESE site, weakening recognition of the upstream 3′ splice site and resulting in the majority of SMN2 transcripts lacking exon 7. As this SMNΔ7 isoform is unstable, and at best, only partially functional, the level of full-length SMN protein is an important modifier of patient disease severity. Antisense technology can therefore be used to promote exon 7 inclusion in the SMN2 transcript.

In a preferred embodiment of the invention the construct comprises an antisense-based system, for example comprising PNA, for inducing the skipping or inclusion of one or more exons in a pre-mRNA, thereby resulting in the expression of functional protein. Accordingly, disclosed is a method of correcting expression of a gene in a human cell having a muscle disease or muscular dystrophy phenotype, wherein without correction the gene fails to express functional protein due to one or more mutations, said method comprising delivering to the cell a nucleic acid comprising a sequence capable of targeting a sequence responsible for exon skipping in the mutated pre-mRNA at an exon to be skipped or included, wherein expression is corrected by the PNA inducing exon skipping or inclusion and thereby correcting the expression of said mutated pre-mRNA

The muscle disease or muscular dystrophy may be any muscular disease or dystrophy that is caused by the aberrant expression of a protein. The aberrant protein expression may be as a result of one or more nonsense or frame-shift mutations. The aberrant protein expression may be the result of a mutation that weakens a splice site resulting in the inclusion of an undesirable exon. Alternatively, the mutation may introduce a cryptic splice site resulting in the splicing of an exon that is desired to be included for protein function.

Examples of muscle diseases include Duchenne muscular dystrophy (DMD), myotonic dystrophy, spinal muscular atrophy, limb-girdle muscular dystrophy, facioscapulohumeral muscular dystrophy, congenital muscular dystrophy, oculpharyngeal muscular dystrophy, distal muscular dystrophy and Emery-Dreifuss dystrophy. Where the disease is DMD, the gene for which expression may be corrected is the dystrophin gene. Where the disease is myotonic dystrophy, the gene for which expression may be corrected is the muscle specific chloride channel (ClC-1) gene. Where is disease is spinal muscular atrophy, the gene for which expression may be corrected is the SMN2 gene.

The human cell may be any human cell in which the gene for which expression is to be corrected has one or more mutations. The one or more mutations may be nonsense or frame-shift mutations. The one or more mutations may strengthen a cryptic splice site or may weaken a splice site. The cell has a muscle disease/dystrophy phenotype, i.e. does not produce a particular functional protein. The cell may be taken from a human patient that has a muscle disease/dystrophy. For example, the cell may be taken from a human patient that has DMD, myotonic dystrophy or spinal muscular atrophy.

Nucleic acid such as PNA can be used for the purpose of inducing exon skipping, or alternatively, exon inclusion. More than one exon can be induced to be skipped at a time. This is desirable because there are often numerous exons in a gene that could potentially be mutated resulting in muscle disease/dystrophy. By targeting the skipping of more than one exon it is possible to remove a larger region of potentially mutant mRNA resulting in the expression of a shortened but functional protein. Any number of exons may be skipped provided that the remaining exons are sufficient to result in the expression of suitably functional protein. Accordingly, 1, 2, 3, 4, 5, 6, 7, 8 or more exons may be skipped.

The disclosed method results in the induction of expression of functional protein. Typically, the amount of functional protein expressed in the cell is at least 10% of the amount of functional protein expressed in a cell in which the gene is not mutated. Preferably, the amount of functional protein expressed in a cell is at least 15%, 20%, 25%, 30%, or more preferably, at least 40% or 50% of the amount of functional protein expressed in a cell in which the gene is not mutated. A method for determining the relative amount of functional protein expressed may be any suitable method known in the art, for example Western blotting.

The functional protein that is expressed by the method is preferably capable of performing the function(s) of the corresponding protein expression from a non-mutated gene. The functional protein may not be 100% as effective as the normal protein but is preferably at least 50%, 60%, 70%, 80%, 90% or more preferably, at least 95% as effective as the normal protein. Functional activity may be determined by any method known in the art to the skilled person that is relevant to the protein concerned.

### Therapeutic Treatment

The ability of the constructs of the invention to deliver biologically active compounds to cells, e.g. cardiac and skeletal muscle cells, results in the suitability of the constructs of the invention for therapeutic treatment of disease, such as muscle disease or muscular dystrophy, in a subject having such a disease. As used herein, the term “treatment” is meant to encompass therapeutic, palliative and prophylactic uses.

This method of treatment or diagnosis is suitable for any patient that has, may have, or is suspected of having, a disease, such as a muscle disease or muscular dystrophy. The disease may be caused by a nonsense or frameshift mutation. The aberrant protein expression may be the result of a mutation that weakens a splice site resulting in the inclusion of an unsuitable exon. Alternatively, the mutation may introduce a cryptic splice site resulting in the splicing of an exon that is important for protein function. The muscle disease or muscular dystrophy may be any muscle disease or dystrophy. Examples include Duchenne muscular dystrophy (DMD), myotonic dystrophy and spinal muscular atrophy.

Symptoms of DMD which may be used to determine whether a subject has DMD include progressive muscle wasting (loss of muscle mass), poor balance, frequent falls, walking difficulty, waddling gait, calf pain, limited range movement, muscle contractures, respiratory difficulty, drooping eyelids (ptosis), gonadal atrophy and scoliosis (curvature of the spine). Other symptoms can include cardiomyopathy and arrhythmias.

Symptoms of myotonic dystrophy which may be used to determine whether a subject has myotonic dystrophy include abnormal stiffness of muscles and myotonia (difficulty or inability to relax muscles). Other symptoms of myotonic dystrophy include weakening and wasting of muscles (where the muscles shrink over time), cataracts, and heart problems. Myotonic dystrophy affects heart muscle, causing irregularities in the heartbeat. It also affects the muscles of the digestive system, causing constipation and other digestive problems. Myotonic dystrophy may cause cataracts, retinal degeneration, low IQ, frontal balding, skin disorders, atrophy of the testicles, insulin resistance and sleep apnea.

A muscle disease of muscular dystrophy may be diagnosed on the basis of symptoms and characteristic traits such as those described above and/or on the results of a muscle biopsy, DNA or blood test. Blood tests work by determining the level of creatine phosphokinase (CPK). Other tests may include serum CPK, electromyography and electrocardiography. Muscular dystrophies can also alter the levels of myoglobin, LDH, creatine, AST and aldolase.

The method of treatment or diagnosis can be used to treat a subject of any age. The subject is preferably mammal, such as human. Preferably an individual to be treated or diagnosed is as young as possible and/or before symptoms of the disease or condition develop. For example, it is preferable to treat an individual before muscle damage occurs in order to preserve as much muscle as possible. For example, the age of onset of DMD is usually between 2 and 5 years old. Without treatment, most DMD sufferers die by their early twenties, typically from respiratory disorders. Typically therefore, the age of the subject to be treated for DMD is from 2 to 20 years old. More preferably, the age of the subject to be treated is from 4 to 18, from 5 to 15 or from 8 to 12. Myotonic dystrophy generally affects adults with an age at onset of about 20 to about 40 years. Typically, the age of the subject to be treated for myotonic dystrophy is from 2 to 40 years old. More preferably, the age of the subject to be treated is from 4 to 35, from 8 to 30 or from 12 to 25. Preferably the individual to be treated is asymptomatic.

The constructs of the invention may be used to deliver biologically active compounds into any type of muscle tissue. The target muscle tissue may be skeletal muscle, cardiac muscle, or smooth muscle. In DMD patients, targeting the heart muscle may be preferable in patients with cardiac disease or early cardiac symptoms. Such patients may be preferable to treat because of the early mortality associated with this component of the disease.

Current medications and treatments for muscular dystrophy are limited. Inactivity can worsen the disease. Physical therapy and orthopaedic instruments may be helpful. The cardiac problems that occur with myotonic dystrophy and Emery-Dreifuss muscular dystrophy may require a pacemaker. Conventional methods of coping with the disease include exercise, drugs that slow down or eliminate muscle wasting, anabolic steroids and dietary supplements such as creatine and glutamine. The anti-inflammatory corticosteroid prednisone may be used to improve muscle strength and delay the progression of the disease. Other nutritional supplements and steroids that may be used in the treatment of DMD include deflazacort, albuterol, creatine, anabolic steroids, and calcium blockers. The myotonia occurring in myotonic dystrophy may be treated with medications such as quinine, phenytoin or mexiletine. All of the above treatments are aimed at slowing down the progression of the disease or reducing its symptoms. The treatment of the invention may be administered in combination with any such form of treating or alleviating the symptoms of muscle disease or muscular dystrophy.

### Nucleic Acids and Peptide Nucleic Acid (PNA)

In PNAs, the sugar phosphate backbone of DNA is replaced by an achiral polyamide backbone. PNAs have a high affinity for DNA and RNA and high sequence specificity. They are also highly resistant to degradation, being protease- and nuclease-resistant. PNAs are also stable over a wide pH range.

The nucleic acids (such as PNAs) used in the invention are typically at least 10 bases long, such as at least 12, 14, 15, 18, 20, 23 or 25 or more bases in length. Typically, the nucleic acid is less than 35 bases in length. Such as less than 34, 32, 30 or 28 bases long. Preferably, the nucleic acid will be in the range of 15 to 30 bases long, more preferably 15 to 25 or 20 to 30 bases long. The nucleic acids may be 18 or 25 bases in length.

The AOs are complementary to and selectively hybridise to one or more sequences that are responsible for or contribute to the promotion of exon splicing or inclusion. Such a sequence may be a splice site donor sequence, splice site acceptor sequence, splice site enhancer sequence or splice site silencer sequence. Splice site donor, acceptor and enhancer sequences are involved in the promotion of exon splicing and therefore can be targeting with one or more AOs in order to inhibit exon splicing. Splice site silencers are involved in inhibiting splicing and can therefore be targeted with AOs in order to promote exon splicing.

Splice site donor, acceptor, enhancer and silencer sequences may be located within the vicinity of the 5′ or 3′ end of the exon to be spliced from or, in the case of silencer sequences, included into the final mRNA. Splice site acceptor or donor sequences and splice site enhancer or silencer sequences are either known in the art or can be readily determined. Bioinformatic prediction programmes can be used to identify gene regions of relevance to splicing events as a first approximation. For example, software packages such as RESCUE-ESE, ESEfinder, and the PESX server predict putative ESE sites. Subsequent empirical experimental work, using splicing assays well known in the art, can then be carried out in order to validate or optimise the sequences involved in splicing for each exon that is being targeted.

Any exon in which there is a non-sense or frame-shift inducing mutation may be a potential target for deletion from the pre-mRNA by exon skipping. Any of the exons in the dystrophin gene can be targeted for deletion from the dystrophin pre-mRNA. Preferably, the exons that are targeted for deletion are any of the exons in the human dystrophin gene except for exons 65 to 69, which are essential for protein function. Preferably the exon(s) to be deleted are those that are commonly mutated in DMD, i.e. any of exons 2 to 20 or exons 45 to 53.

Preferably, the patient is tested for which mutation they have in order to determine which exon is to be deleted or included. Preferably, the sequence of the nucleic acid used for exon skipping comprises a sequence that is capable of selectively hybridising to a sequence that spans the exon/intron boundary of the exon to be deleted or included. The exon/intron boundary may be the 3′ or 5′ boundary of the exon to be included or deleted. The exon/intron boundary sequence information for a particular gene may be obtained from any source of sequence information, such as the ensemble database. Sequence information, including the exon/intron boundary locations, for the human and mouse dystrophin genes may be found at the following web links:

### Human:

http://vega.sanger.ac.uk/Homo_{—}sapiens/transview?transcript=OTTRUMT00000056182

### Mouse:

http://vega.sanger.ac.uk/Mus_{—}musculus/transview?transcript=OTTMUST00000043357

The currently known mutations, including point mutations, deletions duplications in the entire human dystrophin gene may be accessed at the following web link: http://www.dmd.nl/DMD_deldup.html

More preferably, the AO sequence is selected from sequences capable of selectively hybridising to the exon/intron boundary sequences provided in Table 1 or homologues thereof. The nomenclature in Table 1 is based upon target species (H, human, M, mouse), exon number, and annealing coordinates as described by Mann et al 2002 (Journal of Gene Medicine, 4: 644-654). The number of exonic nucleotides from the acceptor site is indicated as a positive number, whereas intronic bases are given a negative value. For example, H16A(−06±25) refers to an antisense oligonucleotide for human dystrophin exon 16 acceptor region, at coordinates 6 intronic bases from the splice site to 25 exonic bases into exon 16. The total length of this AO is 31 nucleotides and it covers the exon 16 acceptor site.

TABLE 1
Sequences of exon/intron boundaries in human and mouse
dystrophin pre-mRNA (SEQ ID NO: 2 to 122).

| Nomenclature | Sequence (5′-3′) |
|---|---|
| H2A(+12 + 41) | CCA UUU UGU GAA UGU UUU CUU UUG AAC AUC |
| H3A(+20 + 40) | GUA GGU CAC UGA AGA GGU UCU |
| H4A(+11 + 40) | UGU UCA GGG CAU GAA CUC UUG UGG AUC CUU |
| H5A(+25 + 55) | UCA GUU UAU GAU UUC CAU CUA CGA UGU CAG U |
| H6A(+69 + 91) | UAC GAG UUG AUU GUC GGA CCC AG |
| H7A(+45 + 67) | UGC AUG UUC CAG UCG UUG UGU GG |
| H9A(−06 + 23) | CCC UGU GCU AGA CUG ACC GUG AUC UGC AG |
| H12A(+52 + 75) | UCU UCU GUU UUU GUU AGC CAG UCA |
| H13A(+77 + 100) | CAG CAG UUG CGU GAU CUC CAC UAG |
| H14A(+32 + 61) | GUA AAA GAA CCC AGC GGU CUU CUG UCC AUC |
| H15A(+48 + 71) | UCU UUA AAG CCA GUU GUG UGA AUC |
| H16A(−12 + 19) | CUA GAU CCG CUU UUA AAA CCU GUU AAA ACA A |
| H18A(+24 + 53) | CAG CUU CUG AGC GAG UAA UCC AGC UGU GAA |
| HM19A(+35 + 65) | GCC UGA GCU GAU CUG CUG GCA UCU UGC AGU U |
| H22A(+125 + 146) | CUG CAA UUC CCC GAG UCU CUG C |
| H23A(+69 + 98) | CGG CUA AUU UCA GAG GGC GCU UUC UUC GAC |
| H24A(+51 + 73) | CAA GGG CAG GCC AUU CCU CCU UC |
| H25A(+95 + 119) | UUG AGU UCU GUC UCA AGU CUC GAA G |
| H27A(+82 + 106) | UUA AGG CCU CUU GUG CUA CAG GUG G |
| H28A(+99 + 124) | CAG AGA UUU CCU CAG CUC CGC CAG GA |
| H29A(+57 + 81) | UCC GCC AUC UGU UAG GGU CUG UGC C |
| H30A(+25 + 50) | UCC UGG GCA GAC UGG AUG CUC UGU UC |
| H31D(+03 − 22) | UAG UUU CUG AAA UAA CAU AUA CCU G |
| H32A(44 + 73) | CUU GUA GAC GCU GCU CAA AAU UGG CUG GUU |
| H33A(+64 + 88) | CCG UCU GCU UUU UCU GUA CAA UCU G |
| H35A(+24 + 53) | UCU GUG AUA CUC UUC AGG UGC ACC UUC UGU |
| H37A(+134 + 157) | UUC UGU GUG AAA UGG CUG CAA AUC |
| H38A(+88 + 112) | UGA AGU CUU CCU CUU UCA GAU UCA C |
| H39A(+62 + 91) | UUU CCU CUC GCU UUC UCU CAU CUG UGA UUC |
| H41A(+44 + 69) | CAA GCC CUC AGC UUG CCU ACG CAC UG |
| H42A(−4 + 23) | AUC GUU UCU UCA CGG ACA GUG UGC UGG |
| H47A(−06 + 24) | CAG GGG CAA CUC UUC CAC CAG UAA CUG AAA |
| H49A(−11 + 16) | CUG CUA UUU CAG UUU CCU GGG GAA AAG |
| H51A(+66 + 90) | ACA UCA AGG AAG AUG GCA UUU CUA G |
| H52A(+12 + 41) | UCC AAC UGG GGA CGC CUC UGU UCC AAA UCC |
| H53A(+39 + 69) | CAU UCA ACU GUU GCC UCC GGU UCU GAA GGU G |
| H72A(+02 + 28) | GUG UGA AAG CUG AGG GGA CGA GGC AGG |
| H74A(+48 + 72) | CGA GGC UGG CUC AGG GGG GAG UCC U |
| H75A(+34 + 58) | GGA CAG GCC UUU AUG UUC GUG CUG C |
| H77A(+16 + 42) | CUG UGC UUG UGU CCU GGG GAG GAC UGA |
| H78A(+04 + 29) | UCU CAU UGG CUU UCC AGG GGU AUU UC |
| H11A(+75 + 97) | CAU CUU CUG AUA AUU UUC CUG UU |
| H21A(+86 + 108) | GUC UGC AUC CAG GAA CAU GGG UC |
| H36A(+22 + 51) | UGU GAU GUG GUC CAC AUU CUG GUC AAA AGU |
| H40A(−5 + 17) | CUU UGA GAC CUC AAA UCC UGU U |
| H43A(+101 + 120) | GGA GAG AGC UUC CUG UAG CU |
| H44A(+61 + 84) | UGU UCA GCU UCU GUU AGC CAC UGA |
| H46A(+107 + 137) | CAA GCU UUU CUU UUA GUU GCU GCU CUU UUC C |
| H48A(−07 + 23) | UUC UCA GGU AAA GCU CUG GAA ACC UGA AAG |
| H57A(−12 + 18) | CUG GCU UCC AAA UGG GAC CUG AAA AAG AAC |
| H60A(+37 + 66) | CUG GCG AGC AAG GUC CUU GAC GUG GCU CAC |
| H61A(+10 + 40) | GGG CUU CAU GCA GCU GCC UGA CUC GGU CCU C |
| H68A(+22 + 48) | CAU CCA GUC UAG GAA GAG GGC CGC UUC |
| H70A(+98 + 121) | CCU CUA AGA CAG UCU GCA CUG GCA |
| H71A(−03 + 21) | AAG UUG AUC AGA GUA ACG GGA CUG |
| H73A(+06 + 30) | GAU CCA UUG CUG UUU UCC AUU UCU G |
| H26A(−07 + 19) | CCU CCU UUC UGG CAU AGA CCU UCC AC |
| H45A(−06 + 20) | CCA AUG CCA UCC UGG AGU UCC UGU AA |
| H50A(+02 + 30) | CCA CUC AGA GCU CAG AUC UUC UAA CUU CC |
| H55A(+141 + 160) | CUU GGA GUC UUC UAG GAG CC |
| H56A(+102 + 126) | GUU AUC CAA ACG UCU UUG UAA CAG G |
| H58A(+21 + 45) | ACU CAU GAU UAC ACG UUC UUU AGU U |
| H59A(−06 + 16) | UCC UCA GGA GGC AGC UCU AAA U |
| H62A(+8 + 34) | GAG AUG GCU CUC UCC CAG GGA CCC UGG |
| H63A(+11 + 35) | UGG GAU GGU CCC AGC AAG UUG UUU G |
| H64A(+47 + 74) | GCA AAG GGC CUU CUG CAG UCU UCG GAG |
| H66A(−8 + 19) | GAU CCU CCC UGU UCG UCC CCU AUU AUG |
| H67A(+22 + 47) | GCG CUG GUC ACA AAA UCC UGU UGA AC |
| H69A(−06 + 18) | UGC UUU AGA CUC CUG UAC CUG AUA |
| H76A(+53 + 79) | GCU GAC UGC UGU CGG ACC UCU GUA GAG |
| H8A(−06 + 18) | GAU AGG UGG UAU CAA CAU CUG UAA |
| H10A(−05 + 16) | CAG GAG CUU CCA AAU GCU GCA |
| H10A(+98 + 119) | UCC UCA GCA GAA AGA AGC CAC G |
| H17A(−07 + 16) | UGA CAG CCU GUG AAA UCU GUG AG |
| H20A(+44 + 71) | CUG GCA GAA UUC GAU CCA CCG GCU GUU C |
| H20A(147 + 168) | CAG CAG UAG UUG UCA UCU GCU C |
| H34A(+46 + 70) | CAU UCA UUU CCU UUC GCA UCU UAC G |
| H34A(+95 + 120) | AUC UCU UUG UCA AUU CCA UAU CUG UA |
| H54A(+67 + 89) | UCU GCA GAA UAA UCC CGG AGA AG |
| H65A(−11 + 14) | GCU CAA GAG AUC CAC UGC AAA AAA C |
| H65A(+63 + 87) | UCU GCA GGA UAU CCA UGG GCU GGU C |
| H65D(+15 − 11) | GCC AUA CGU ACG UAU CAU AAA CAU UC |
| H16A(−17 + 08) | UUU AAA ACC UGU UAA AAC AAG AAA G |
| H16A(−12 + 19) | CUA GAU CCG CUU UUA AAA CCU GUU AAA ACA A |
| H16A(−06 + 19) | CUA GAU CCG CUU UUA AAA CCU GUU A |
| H16A(−06 + 25) | UCU UUU CUA GAU CCG CUU UUA AAA CCU GUU A |
| H16A(−07 + 13) | CCG CUU UUA AAA CCU GUU AA |
| H16A(+01 + 25) | UCU UUU CUA GAU CCG CUU UUA AAA C |
| H16A(+06 + 30) | CUU UUU CUU UUC UAG AUC CGC UUU U |
| H16A(+11 + 35) | GAU UGC UUU UUC UUU UCU AGA UCC G |
| H16A(+12 + 37) | UGG AUU GCU UUU UCU UUU CUA GAU CC |
| H16A(+45 + 67) | GAU CUU GUU UGA GUG AAU ACA GU |
| H16A(+87 + 109) | CCG UCU UCU GGG UCA CUG ACU UA |
| H16A(+92 + 116) | CAU GCU UCC GUC UUC UGG GUC ACU G |
| H16A(+105 + 126) | GUU AUC CAG CCA UGC UUC CGU C |
| H16D(+11 − 11) | GUA UCA CUA ACC UGU GCU GUA C |
| H16D(+05 − 20) | UGA UAA UUG GUA UCA CUA ACC UGU G |
| H46A(+107 + 137) | CAA GCU UUU CUU UUA GUU GCU GCU CUU UUC C |
| H51A(−01 + 25) | ACC AGA GUA ACA GUC UGA GUA GGA GC |
| H51A(+61 + 90) | ACA UCA AGG AAG AUG GCA UUU CUA GUU UGG |
| H51A(+66 + 90) | ACA UCA AGG AAG AUG GCA UUU CUA G |
| H51A(+66 + 95) | CUC CAA CAU CAA GGA AGA UGG CAU UUC UAG |
| H51A(+111 + 134) | UUC UGU CCA AGC CCG GUU GAA AUC |
| H51A(+175 + 195) | CAC CCA CCA UCA CCC UCU GUG |
| H51A(+199 + 220) | AUC AUC UCG UUG AUA UCC UCA A |
| H51D(+08 − 17) | AUC AUU UUU UCU CAU ACC UUC UGC U |
| H51D(+16 − 07) | CUC AUA CCU UCU GCU UGA UGA UC |
| H53A(−07 + 18) | GAU UCU GAA UUC UUU CAA CUA GAA U |
| H53A(−12 + 10) | AUU CUU UCA ACU AGA AUA AAA G |
| H53A(+23 + 47) | CTG AAG GTG TTC TTG TAC TTC ATC C |
| H53A(+39 + 62) | CUG UUG CCU CCG GUU CUG AAG GUG |
| H53A(+39 + 69) | CAU UCA ACU GUU GCC UCC GGU UCU GAA GGU G |
| H53A(+45 + 69) | CAU UCA ACU GUU GCC UCC GGU UCU G |
| H53A(+124 + 145) | UUG GCU CUG GCC UGU CCU AAG A |
| H53A(+151 + 175) | GUA UAG GGA CCC UCC UUC CAU GAC U |
| H53D(+09 − 18) | GGU AUC UUU GAU ACU AAC CUU GGU UUC |
| H53D(+14 − 07) | UAC UAA CCU UGG UUU CUG UGA |
| M23D(+07 − 18) | GGC CAA ACC UCG GCU UAC CUG AAA U |
| M23D(+02 − 18) | GGC CAA ACC UCG GCU UAC CU |
| M23D(+12 − 18) | GGC CAA ACC UCG GCU UAC CUG AAA UUU UCG |
| M23D(+07 − 23) | UUA AAG GCC AAA CCU CGG CUU ACC UGA AAU |

Examples of preferred AO sequences capable of inducing the splicing of exon 7a in the mouse ClC-1 gene are sequences capable of selectively hybridising to the 3′ or 5′ splice sites of exon 7a. Such preferred AO sequences may be capable of specifically hybridising to a sequence in Table 2 or a homologue thereof.

TABLE 2
Sequences of exon/intron boundaries in the mouse CIC-1
pre-mRNA for mouse exon 7a (SEQ ID NO: 123 and 124).

| Nomenclature | Sequence (5′-3′) |
|---|---|
| M7a (−17 + 14) | GUG CUU CUC UGU UGC AGA CCG UGC CUG GGC A |
| M7a (+13 − 18) | GCC CCT GAU GGA GGC AAG UUU CAC UUC CUC C |

Typically, only one AO sequence is used to induce or inhibit exon skipping in a cell. However, more than one different AO can be delivered to the sample of human cells or a patient, e.g. a cocktail of 2, 3, 4 or 5 or more different AO sequences can be used to drive exon skipping or inhibit exon skipping in a cell. Such a combination of different AO sequences can be delivered simultaneously, separately or sequentially.

Selective hybridisation means that generally the polynucleotide can hybridize to the relevant polynucleotide, or portion thereof, at a level significantly above background. The signal level generated by the interaction between the polynucleotides is typically at least 10 fold, preferably at least 100 fold, as intense as interactions between other polynucleotides. The intensity of interaction may be measured, for example, by radiolabelling the polynucleotide, e.g. with ³²P. Selective hybridisation is typically achieved using conditions of medium to high stringency (for example 0.03M sodium chloride and 0.003M sodium citrate at from about 50° C. to about 60° C.).

PNAs are produced synthetically using any known technique in the art. PNA is a DNA analog in which a polyamide backbone replaces the traditional phosphate ribose ring of DNA. Despite a radical change to the natural structure, PNA is capable of sequence-specific binding to DNA or RNA. Characteristics of PNA include a high binding affinity to complementary DNA or RNA, a destabilizing effect caused by single-base mismatch, resistance to nucleases and proteases, hybridization with DNA independent of salt concentration and triplex formation with homopurine DNA.

Panagene™ has developed its proprietary Bts PNA monomers (Bts; benzothiazole-2-sulfonyl group) and proprietary oligomerisation process. The PNA oligomerisation using Bts PNA monomers is composed of repetitive cycles of deprotection, coupling and capping. Panagene's patents to this technology include U.S. Pat. No. 6,969,766, U.S. Pat. No. 7,211,668, U.S. Pat. No. 7,022,851, U.S. Pat. No. 7,125,994, U.S. Pat. No. 7,145,006 and U.S. Pat. No. 7,179,896.

Delivery using Glucose Analogues

The invention provides a composition for use in delivering a nucleic acid or a conjugate of the invention to a cell. The conjugate may be any of the conjugates mentioned herein, and in one embodiment the conjugate does not comprise a nucleic acid (but comprises another type of biologically active compound instead). The composition comprises a glucose analogue, preferably at a concentration of 2 to 50%, such as 4 to 20% or 6 to 15%. The glucose analogue is typically a sugar (excluding glucose), and in certain embodiments may be galactose, mannose, fructose, 2-DG, 3-OMG or AMG.

### Homologues

Homologues of polynucleotide and polypeptide sequences are referred to herein. Such homologues typically have at least 70% homology, preferably at least 80, 90%, 95%, 97% or 99% homology, for example over a region of at least 5, 10, 15, 20, 25 or more contiguous nucleotides or amino acids or over the entire length of the original polynucleotide or polypeptide. The homology may be calculated on the basis of nucleotide or amino acid identity (sometimes referred to as “hard homology”).

For example the UWGCG Package provides the BESTFIT program which can be used to calculate homology (for example used on its default settings) (Devereux et al (1984) Nucleic Acids Research 12, p 387-395). The PILEUP and BLAST algorithms can be used to calculate homology or line up sequences (such as identifying equivalent or corresponding sequences (typically on their default settings), for example as described in Altschul S. F. (1993) J Mol Evol 36:290-300; Altschul, S, F et al (1990) J Mol Biol 215:403-10.

Software for performing BLAST analyses is publicly available through the National Center for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two polynucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

The homologous sequence typically differs by at least 1, 2, 5, 10, 20 or more mutations (which may be substitutions, deletions or insertions of nucleotides or amino acids). These mutations may be measured across any of the regions mentioned above in relation to calculating homology.

### Delivery/Administration

The constructs of the invention may be administered by any suitable means. Administration to a human or animal subject may be selected from parenteral, intramuscular, intracerebral, intravascular, subcutaneous, or transdermal administration. Typically the method of delivery is by injection. Preferably the injection is intramuscular or intravascular (e.g. intravenous). A physician will be able to determine the required route of administration for each particular patient.

The constructs are preferably delivered as a composition. The composition may be formulated for parenteral, intramuscular, intracerebral, intravascular (including intravenous), subcutaneous, or transdermal administration. For example, uptake of nucleic acids by mammalian cells is enhanced by several known transfection techniques, for example, those that use transfection agents. The formulation that is administered may contain such agents. Examples of these agents include cationic agents (for example calcium phosphate and DEAE-dextran) and lipofectants (for example lipofectam™ and transfectam™).

Compositions for parenteral administration may include sterile aqueous solutions which may also contain buffers, diluents and other suitable additives. In some cases it may be more effective to treat a patient with a construct of the invention in conjunction with other disease therapeutic modalities (such as those described herein) in order to increase the efficacy of the treatment.

The constructs of the invention may be formulated in a pharmaceutical composition, which may include pharmaceutically acceptable carriers, thickeners, diluents, buffers, preservatives, surface active agents, neutral or cationic lipids, lipid complexes, liposomes, penetration enhancers, carrier compounds and other pharmaceutically acceptable carriers or excipients and the like in addition to the construct. The composition may comprise other active agents that are used in therapy (e.g. anti-inflammatories for DMD therapy).

The constructs may be used in combination with other methods of molecular therapy. For example, the construct may be delivered in combination (simultaneously, separately or sequentially) with a gene or partial gene encoding the protein which is mutated in the individual. For example, the gene may be the full-length or partial sequence of the dystrophin gene in cases of DMD. Gene therapy targeting the myostatin gene or its receptor may also be used in conjunction with the construct(s) in order to increase muscle mass and thereby restore strength in any remaining muscle. Gene delivery may be carried out by any means, but preferably via a viral vector.

Formulations for parenteral administration may include sterile aqueous solutions which may also contain buffers, liposomes, diluents and other suitable additives. Pharmaceutical compositions comprising the construct provided herein may include penetration enhancers in order to enhance the delivery of the construct. Penetration enhancers may be classified as belonging to one of five broad categories, i.e. fatty acids, bile salts, chelating agents, surfactants and non-surfactants. One or more penetration enhancers from one or more of these broad categories may be included.

Various fatty acids and their derivatives which act as penetration enhancers include, for example, oleic acid, lauric acid, capric acid, myristic acid, palmitic acid, stearic acid, linoleic acid, linolenic acid, dicaprate, tricaprate, recinleate, monoolein (1-monooleoyl-rac-glycerol), dilaurin, caprylic acid, arachidonic acid, glyceryl 1-monocaprate, 1-dodecylazacycloheptan-2-one, acylcarnitines, acylcholines, mono- and di-glycerides and physiologically acceptable salts thereof (i.e. oleate, laurate, caprate, myristate, palmitate, stearate, linoleate, etc).

Various natural bile salts, and their synthetic derivatives, act as penetration enhancers. Thus, the term “bile salt” includes any of the naturally occurring components of bile as well as any of their synthetic derivatives.

Complex formulations comprising one or more penetration enhancers may be used. For example, bile salts may be used in combination with fatty acids to make complex formulations. Chelating agents include, but are not limited to, disodium ethylenediaminetetraacetate (EDTA), citric acid, salicylates (e.g. sodium salicylate, 5-methoxysalicylate and homovanilate), N-acyl derivatives of collagen, laureth-9 and N-amino acyl derivatives of beta-diketones (enamines). Chelating agents have the added advantage of also serving as DNase inhibitors.

Surfactants include, for example, sodium lauryl sulfate, polyoxyethylene-9-lauryl ether and polyoxyethylene-20-cetyl ether and perfluorochemical emulsions, such as FC-43. Non-surfactants include, for example, unsaturated cyclic ureas, 1-alkyl- and 1-alkenylazacyclo-alkanone derivatives and non-steroidal anti-inflammatory agents such as diclofenac sodium, indomethacin and phenylbutazone.

A “pharmaceutically acceptable carrier” (excipient) is a pharmaceutically acceptable solvent, suspending agent or any other pharmacologically inert vehicle for delivering one or more nucleic acids to a subject. The pharmaceutically acceptable carrier may be liquid or solid and is selected with the planned manner of administration in mind so as to provide for the desired bulk, consistency etc when combined with a nucleic acid and the other components of a given pharmaceutical composition. Typical pharmaceutically acceptable carriers include, but are not limited to, binding agents (e.g. pregelatinised maize starch, polyvinylpyrrolidone or hydroxypropyl methylcellulose, etc); fillers (e.g. lactose and other sugars, microcrystalline cellulose, pectin, gelatin, calcium sulfate, ethyl cellulose, polyacrylates or calcium hydrogen phosphate, etc); lubricants (e.g. magnesium stearate, talc, silica, colloidal silicon dioxide, stearic acid, metallic stearates, hydrogenated vegetable oils, corn starch, polyethylene glycols, sodium benzoate, sodium acetate, etc); disintegrates (e.g. starch, sodium starch glycolate, etc); or wetting agents (e.g. sodium lauryl sulphate, etc).

The compositions provided herein may additionally contain other adjunct components conventionally found in pharmaceutical compositions. Thus, for example, the compositions may contain additional compatible pharmaceutically-active materials or may contain additional materials useful in physically formulating various dosage forms of the composition of present invention, such as dyes, flavouring agents, preservatives, antioxidants, opacifiers, thickening agents and stabilizers. However, such materials, when added, should not unduly interfere with the biological activities of the components of the compositions provided herein.

Regardless of the method by which the constructs are introduced into a patient, colloidal dispersion systems may be used as delivery vehicles to enhance the in vivo stability of the construct and/or targeting the construct to a particular organ, tissue or cell type. Colloidal dispersion systems include, but are not limited to, macromolecule complexes, nanocapsules, microspheres, beads and lipid-based systems including oil-in-water emulsions, micelles, mixed micelles, liposomes and lipid:oligonucleotide complexes of uncharacterised structure. A preferred colloidal dispersion system is a plurality of liposomes. Liposomes are microscopic spheres having an aqueous core surrounded by one or more outer layers made up of lipids arranged in a bilayer configuration.

A therapeutically effective amount of construct is administered. The dose may be determined according to various parameters, especially according to the severity of the condition, age, and weight of the patient to be treated; the route of administration; and the required regimen. A physician will be able to determine the required route of administration and dosage for any particular patient. Optimum dosages may vary depending on the relative potency of individual constructs, and can generally be estimated based on EC50s found to be effective in vitro and in in vivo animal models. In general, dosage is from 0.01 mg/kg to 100 mg per kg of body weight. A typical daily dose is from about 0.1 to 50 mg per kg, preferably from about 0.1 mg/kg to 10 mg/kg of body weight, according to the potency of the specific construct, the age, weight and condition of the subject to be treated, the severity of the disease and the frequency and route of administration. Different dosages of the construct may be administered depending on whether administration is by intramuscular injection or systemic (intravenous or subcutaneous) injection. Preferably, the dose of a single intramuscular injection is in the range of about 5 to 20 ug. Preferably, the dose of single or multiple systemic injections is in the range of 10 to 100 mg/kg of body weight.

Due to construct clearance (and breakdown of any targeted molecule), the patient may have to be treated repeatedly, for example once or more daily, weekly, monthly or yearly. Persons of ordinary skill in the art can easily estimate repetition rates for dosing based on measured residence times and concentrations of the construct in bodily fluids or tissues. Following successful treatment, it may be desirable to have the patient undergo maintenance therapy, wherein the construct is administered in maintenance doses, ranging from 0.01 mg/kg to 100 mg per kg of body weight, once or more daily, to once every 20 years.

The invention is illustrated by the following Examples:

### EXAMPLE 1

Muscular dystrophy (MD) refers to a group of more than 30 inherited diseases that cause muscle weakness and muscle loss. Some forms of MD appear in infancy or childhood, while others may not appear until middle age or later. The different muscular dystrophies vary in who they affect and the symptoms. All forms of MD grow worse as the person's muscles get weaker. Some types of MD affect heart muscles, other types affect involuntary muscles and other organs. Most people with MD eventually lose the ability to walk. Muscles, primarily voluntary muscles, become progressively weaker. The most common types of MD are due to a genetic deficiency of the muscle protein dystrophin.

Duchenne muscular dystrophy (DMD) is an X-linked muscle disorder mainly caused by nonsense or frame-shift mutations in the dystrophin gene resulting in a dystrophin deficiency in muscle cells. DMD has an incidence of about 1:3500 newborn males. DMD patients suffer from severe, progressive muscle wasting and most will stop walking by the age of 12 years with about 90% not surviving beyond the age of 20. The milder allelic form of the disease, Becker muscular dystrophy (BMD), is usually caused by in-frame deletions resulting in expression of a shortened, but partially functional protein. BMD patients have milder symptoms and longer life expectancies when compared to DMD patients. The severity of the disease varies, and boys and men with Becker dystrophy have a longer life expectancy than those with Duchenne. The severity and rate of progression of Becker dystrophy depends on how much dystrophin is made and how well it functions in the muscles.

Myotonic dystrophy, also known as Steinert's disease, is the most common adult form of muscular dystrophy. Its name underscores an unusual symptom found only in this form of dystrophy—myotonia, which is similar to a spasm or stiffening of muscles after use. The disease causes muscle weakness and affects the central nervous system, heart, gastrointestinal tract, eyes (causing cataracts) and endocrine (hormone-producing) glands. Although muscle weakness progresses slowly, this symptom can vary greatly, even among members of a single family. Most often muscle weakness does not hamper daily living for many years after symptoms first occur.

Most often, the onset of Limb-Girdle muscular dystrophy (LGMD) is in adolescence or early adulthood. In the most common forms, the disease causes progressive weakness that starts in the hips and moves to the shoulders. The weakness progresses to include the arms and legs. Within 20 years of onset, walking is difficult, if not impossible.

The word facioscapulohumeral refers to the muscles that move the face, scapula (shoulder blade) and humerus (upper arm bone). Common early signs of Facioscapulohumeral muscular dystrophy (FSH) are a forward sloping of the shoulders as well as difficulty raising the arms over the head and closing the eyes. Progression is slow, with long periods of stability interspersed with shorter periods of rapid muscle deterioration and increased weakness. The muscles of the face and shoulder area are the first affected. The weakness spreads to the muscles of the abdomen, feet, upper arms, pelvic area and lower arms, usually in that order. The disease ranges in severity from very mild to considerably disabling, with impairment of walking, chewing, swallowing and speaking. About half of those with the disorder retain the ability to walk throughout their lives.

Congenital muscular dystrophy (CMD) is a group of diseases in which symptoms can be noted from birth. One form that has been clearly described is Fukuyama congenital muscular dystrophy. This disorder involves severe weakness of the facial and limb muscles and a generalized lack of muscle tone, usually appearing before 9 months. Joint contractures are common. Brain abnormalities are also present, and most children have severe mental and speech problems. Seizures are often part of the disease, and medications are prescribed for these. Physical therapy is needed to minimize the contractures. Another form of congenital dystrophy seems to be related to a deficiency or malfunction of the protein merosin, which normally lies outside muscle cells and links them to the surrounding tissue. The disorder is similar to Fukuyama dystrophy, with muscle weakness evident at birth or in the first few months of life, severe and early contractures and often joint deformities. This disorder has been tentatively named congenital muscular dystrophy with merosin deficiency.

Oculpharyngeal, meaning eye and throat, muscular dystrophy (OPMD) usually starts with drooping of the eyelids, most often in the 40s or 50s. This is followed by other signs of eye and facial muscle weakness, as well as by difficulty in swallowing. The later stages of this slowly progressive disease may include weakness in the pelvic and shoulder muscles. Swallowing problems can lead to choking and recurrent pneumonia.

Distal muscular dystrophy (DD) is actually a group of rare muscle diseases, which have in common weakness and wasting of the distal muscles of the forearms, hands, lower legs and feet. A type of distal dystrophy called Welander is inherited in an autosomal dominant pattern and affects the hands first. Another type, known as Markesbery-Griggs, is autosomal dominant in its inheritance and affects the front of the lower legs first, as does Nonaka dystrophy. Miyoshi dystrophy, caused by a gene defect on chromosome 2, is autosomal recessive and affects the back of the lower legs first.

Emery-Dreifuss dystrophy (EDMD) is a rare form of muscular dystrophy. Muscle weakness and wasting generally start in the shoulders, upper arms and lower legs. Weakness may later spread to involve the muscles of the chest and pelvic area. Contractures appear early in the disease, usually involving the ankle and elbow. Unlike other forms of muscular dystrophy, contractures in Emery-Dreifuss dystrophy often appear before the person experiences significant muscle weakness. Physical therapy is beneficial in minimizing the contractures. Life-threatening heart problems are a common part of this disorder. The heart problems are electrical and can be treated with a cardiac pacemaker. These problems can even occur in females who do not have the disease but are carriers, so sisters and mothers of boys with Emery-Dreifuss should be examined. The skeletal muscle weakness is less severe than it is in some other dystrophies, such as Duchenne. Emery-Dreifuss dystrophy is caused by a defect in the gene on the X chromosome that codes for the protein emerin.

Duchenne muscular dystrophy is therefore one of many disease states that might benefit from the development of peptides that could deliver molecules to cardiac and skeletal muscle cells.

### Materials and Methods

### Animals

Three age groups of mdx mice were used: 20-21 days (referred to as 3 weeks; five mice for each test and control groups), 2-month old (five mice for test groups and control groups), and 5-6 months (referred to 6 months, six mice for each testing and control group). Mice were killed by cervical dislocation at desired time points, and muscles were snap-frozen in liquid nitrogen-cooled isopentane and stored at −80° C.

### PNA-Peptide Conjugates

Details of PNA-peptide conjugates are shown in Table 3. Conjugations of peptide with PNA were synthesized by disulphide-bridge linkage. All the PNA-peptide conjugates were synthesized by EuroGentec (LIEGE Science Park, Belgium). The PNA AO sequence against the boundary sequences of exon and intron 23 of the dystrophin gene was 5′-ggccaaaccteggcttacct-3′, and designated as PNA.

TABLE 3
Peptide-PNA structures

| Peptide | | Peptide |
|---|---|---|
| names | Sequence (from N-terminal to C-terminal) | length |
| TAT | YGRKKRRQRRRP-S-S-Lggccaaacctcggcttacct | 12 |
| MSP | ASSLNIA-S-S-Lggccaaacctcggcttacct | 7 |
| AAV6 | TVAVNLQSSSTDPATGDVHVM-S-S-Lggccaaacctcggcttacct | 21 |
| AAV8 | IVADNLQQQNTAPQIGTVNSQ-S-S-Lggccaaacctcggcttacct | 21 |

Capital letters are amino acid residues, lower letters are PNA nucleotides; —S—S-refers to a disulphide bridge; L means AEEA linker.

### Cell Culture and Transfection

The H₂K mdx myoblasts were cultured at 33° C. under a 10% CO₂/90% air atmosphere in high-glucose DMEM supplemented with 20% fetal calf serum, 0.5% chicken embryo extract (PAA laboratories Ltd, Yeovil, UK), and 20 units/ml γ-interferon (Roche applied science, Penzberg, Germany). Cells were then treated with trypsin and plated at 2×10⁴ cells per well in 24-well plates coated with 200 ug/ml gelatine (Sigma). H₂K mdx cells were transfected 24 h after trypsin treatment in a final volume of 0.5 ml of antibiotic- and serum-free Opti-MEM (Life Technologies). Each well was treated with 250 nM of PNA-peptide complexed with corresponding amounts of lipofectin (weight ratio 1:2=oligolipofectin) (Life Technologies) according to the supplier's instructions. After 4 h of incubation, the transfection medium was replaced with DMEM supplemented medium.

### RNA Extraction and Nested RT-PCR Analysis

Cells were transfected as triplicate wells with Lipofectin-Oligonucleotide complexes and total cellular RNA was then extracted 24 h after transfection with RNAeasy mini kit (Qiagen) and 200 ng of RNA template was used for 20 μl RT-PCR with OneStep RT-PCR kit (Qiagen, West Sussex, UK). The primer sequences for the initial RT-PCR were Exon20Fo 5′-CAGAATTCTGCCAATTGCTGAG-3′ (SEQ ID NO: 125) and Ex26Ro 5′-TTCTTCAGCTTGTGTCATCC-3′ (SEQ ID NO: 126) for amplification of mRNA from exons 20 to 26. The cycling conditions were 95° C. for 30 sec, 55° C. for lmin, and 72° C. for 2 min for 30 cycles. RT-PCR product (1 μl) was then used as the template for secondary PCR performed in 25 μl with 0.5 unit TaqDNA polymerase (Invitrogen). The primer sequences for the second round were:

| (SEQ ID NO: 127) | |
|---|---|
| Ex20Fi | 5′-CCCAGTCTACCACCCTATCAGAGC-3′ |
| and | |
| (SEQ ID NO: 128) | |
| Ex2Ri | 5′-CCTGCCTTTAAGGCTTCCTT-3′. |

The cycling conditions were 95° C. for 1 min, 57° C. for lmin, and 72° C. for 2 min for 25 cycles. Products were examined by electrophoresis on a 2% agarose gel.

### Intramuscular Injection of PNA-Peptide Conjugates

One tibialis anterior (TA) muscle of each experimental mdx mouse was injected with a 40 μl dose (3-week-old group injected with 10 μl) of PNA-peptide conjugates with saline at a final concentration of 125 μg/ml, and the contralateral muscle was injected with saline. The animals were sacrificed at various time points after injection, the muscles were removed and snap-frozen in liquid nitrogen-cooled isopentane and stored at −80° C. To examine the dose-response profile, 2-month old mdx mice received the injections of 10 μg and 20 μg of PNA-peptide conjugates.

### Immunohistochemistry and Histology

Sections of 8 μm were cut from at least two-thirds of muscle length of TA muscles at 100 μm intervals. The sections were then examined for dystrophin expression with a polyclonal antibody 2166 against the dystrophin carboxyl-terminal dystrophin. The maximum number of dystrophin-positive fibres in one section was counted using the Zeiss AxioVision fluorescence microscope. The intervening muscle sections were collected either for Western blot or as serial sections for immunohistochemistry. Polyclonal antibodies were detected by goat-anti-rabbit IgGs Alexa Fluro 594 (Molecular probe). Routine H&E staining was used to examine overall muscle morphology and assess the level of infiltrating mononuclear cells.

### Protein Extraction and Western Blot

The collected sections were placed in a 1.5 ml polypropylene eppendorf tube (Anachem, Bedfordshire, UK) on dry ice. The tissue sections were lysed with 150 μl protein extraction buffer containing 125 mM Tris-HCl (pH6.8), 10% SDS, 2M urea, 20% glycerol and 5% 2-mercaptoeethanol. The mixture was boiled for 5 min and centrifuged. The supernatant was collected and the protein concentration was quantified by BCA assay (Sigma). Protein (5 μg) from normal C57BL6 mice as a positive control and 50 μg of protein from muscles of treated or untreated mdx mice were loaded onto SDS-PAGE gels (4% stacking, 6% resolving). Samples were electrophoresed for 4 h at 80 mA and transferred to nitrocellulose overnight at 50V at 4° C. The membrane was then washed and blocked with 5% skimmed milk and probed with DYS1 (monoclonal antibody against dystrophin R8 repeat, 1:200, NovoCastra) overnight. The bound primary antibody was detected by horseradish peroxidase-conjugated rabbit anti-mouse IgGs and ECL Western Blotting Analysis system (Amersham Pharmacia Biosciences). The intensity of the bands obtained from treated mdx muscles was measured by Image J and compared with that from normal muscle of C57BL6 mice.

### Statistical Analysis

All data are reported as mean value±SEM. Statistical differences between treatment groups were evaluated by SigmaStat (Systat Software Inc, UK).

### Results

### Evaluation of PNA-Peptide Conjugates

The possible toxicity of the PNA-peptide conjugates in H2K mdx cells was examined using a WST-1 assay, which measures the metabolic activity of viable cells. Cells were grown in 96-well microplates overnight and treated with unmodified PNA as a control or PNA-peptide conjugates for 12 hours, and then incubated with WST-1 for approximately 4 hours. During this incubation period, viable cells convert WST-1 to a water-soluble formazan dye which is then quantified using an ELISA plate reader.

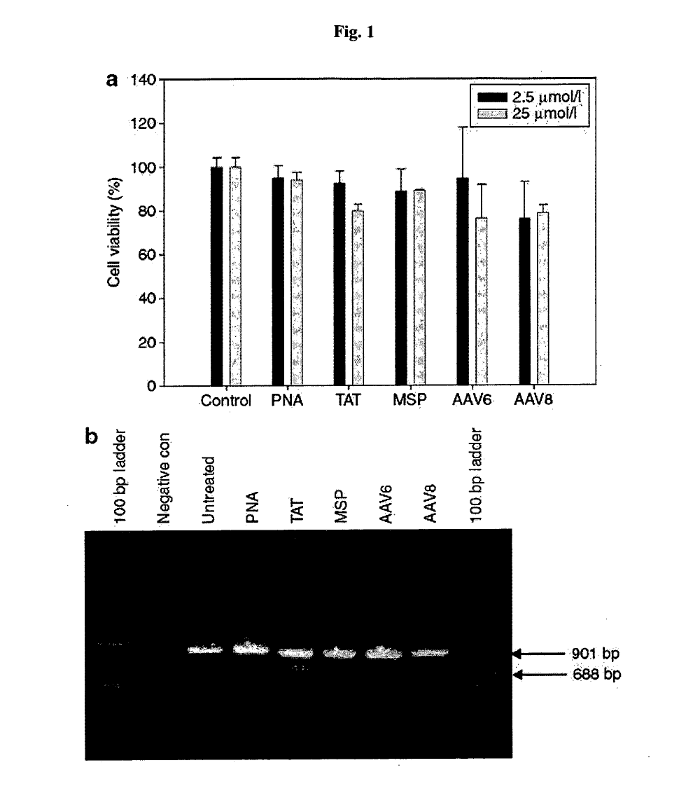

Toxicity or inhibition of cell proliferation was not observed when the cells were treated with the unmodified PNA or PNA-peptide conjugates at concentrations ranging from 2.5 μM to 25 μM, which are 10 and 100 fold higher concentrations respectively than that used for cell transfection experiments with unmodified PNA (FIG. 1A). Lack of toxicity is significant because it supports the conclusion that the PNA-peptide conjugates do not non-specifically block critical cellular processes even though the charged peptides could promote non-specific associations with DNA and RNA.

The effect of the PNA-peptide conjugates on exon skipping in vitro was then examined. H2K mdx cells were transfected using identical transfection conditions as for PNA alone. Exon-skipping was verified by RT-PCR, as shown in FIG. 1B. The efficient removal of exon 23 from the dystrophin transcript was shown with the TAT and AAV8 conjugates at a concentration of 250 nm, but not with the MSP or AAV6 conjugates, even though all of the PNA-peptide conjugates had an identical PNA sequence.

### Evaluation of PNA-Peptide Conjugates by Intramuscular Injection

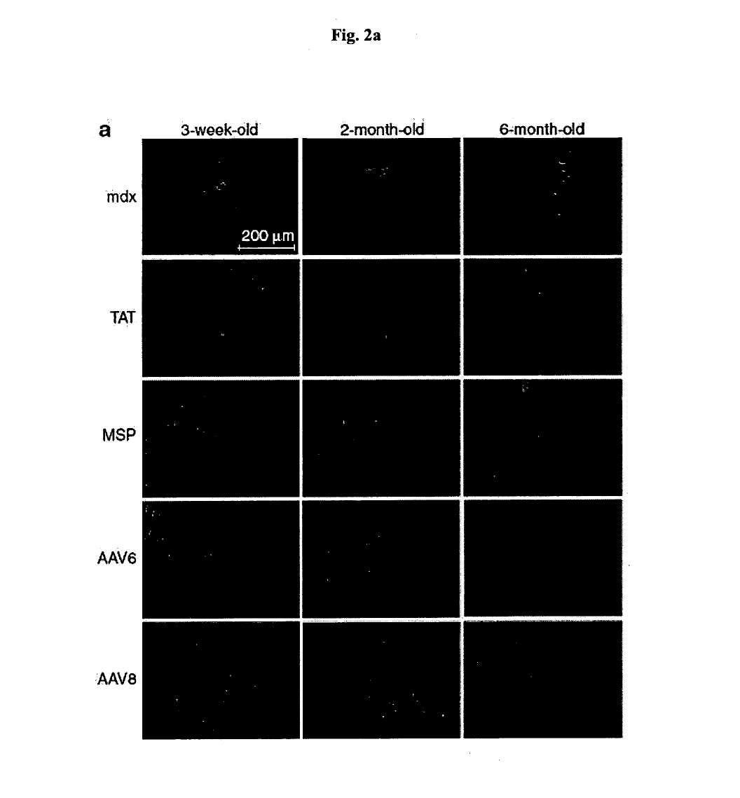

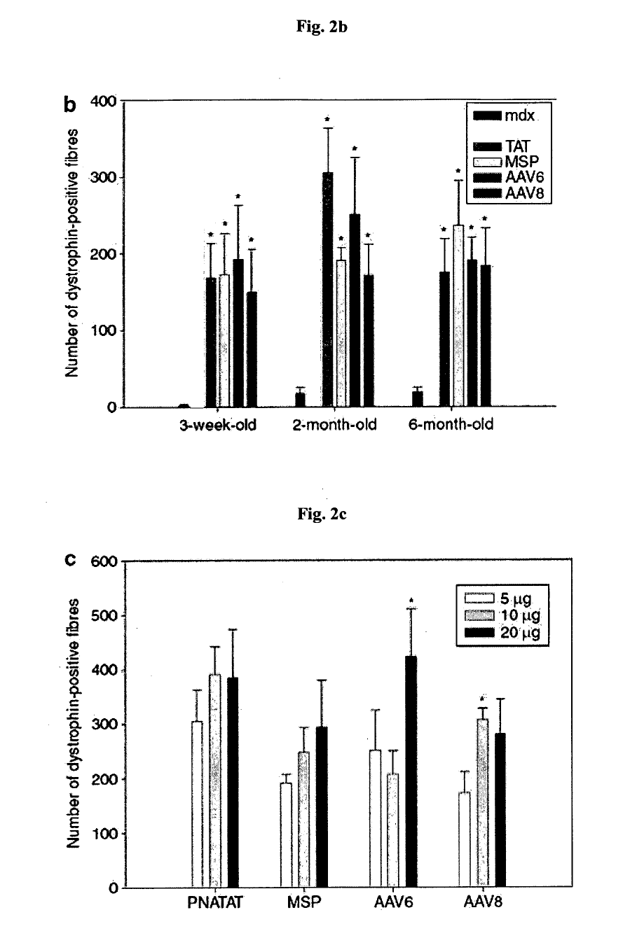

The inventors' previous in vitro and in vivo studies have shown that the neutral chemistry AOs, for example PMOs, are relatively inefficient for cellular uptake and exon skipping in in vitro studies. However, they can be effectively delivered in animal models and induce specific exon skipping with high efficiency in vivo. The inventors therefore examined all of the PNA-peptide conjugates for their exon skipping potential by intramuscular injection of 2-month old mdx mice. A single injection of 5 μg TAT into TA muscles produced clear exon-skipping as evidenced by a high number of dystrophin-positive fibres (average value is 305±58) within muscle transverse sections distributed broadly, but not uniformly throughout the muscle cross section. A large number of dystrophin-positive fibres was observed in muscles treated with TAT, MSP, AAV6 and AAV8 peptide conjugates and focal areas with strong dystrophin-positive muscle fibres were observed with MSP, AAV6 and TAT (FIG. 2).

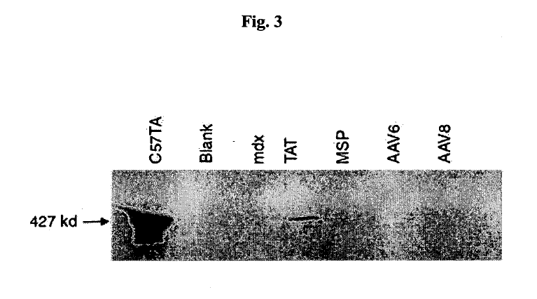

The expression of dystrophin in TA muscles injected with PNA-peptide conjugates was also demonstrated by Western blot. There was as high as 4% of the normal level induced when compared to the normal C57 muscles (FIG. 3).

To explore whether animal age affected the delivery efficiency of PNA-peptide conjugates, age-course experiments were conducted using 3-week and 6-month old mdx mice. Quantification of dystrophin-positive fibres in TA muscle transverse sections showed no difference between 3-week and 6-month old groups after single intramuscular injections of 5 μg PNA-peptide conjugates. Interestingly, MSP showed pronounced improvement of the expression of dystrophin in the 6-month old mdx mice (237±58) as shown in FIG. 2A. In comparison with the treatments in 2-month old mdx mice, TAT and AAV6 demonstrated slightly fewer dystrophin-positive fibres in the 3-week and 6-month old mdx mice.

To further examine the dose-dependent characteristics, 2-month old mdx mice were injected intramuscularly with 5 μg, 10 μg and 20 μg PNA-peptide conjugates, respectively. Immunohistochemical results showed that higher doses increased the efficiency of exon-skipping for all PNA-peptide conjugates (FIG. 2C), but the range of increase varied considerably. The increase in dystrophin induction was not significant with MSP between the three doses, but significant with AAV6, showing average of 424 (±86) dystrophin-positive fibres per TA muscle using a 20 μg injection compared to only 251 in 5 μg (P<0.05). Surprisingly, a bug injection of AAV8 induced markedly more dystrophin-positive fibres (308±20) than a 5 μg injection (171±40, P<0.05), but no further increase after a 20 μg injection (282±63). The variable dose responses with the different PNA-peptide conjugates are not fully understood, but could possibly be explained by the different peptide structures and uptake pathways.

### EXAMPLE 2

Here we aim to evaluate the potential of PNA AOs as splice correcting therapeutic agents for DMD, by studying their activity under a range of conditions in mdx mice carrying a nonsense mutation in exon 23 of the dystrophin gene. PNAs and PNA-pepide conjugates were studied following intramuscular delivery into adult (6-8 week old) mdx tibialis anterior (TA) muscles or via systemic intravenous tail vein delivery in mdx mice unless otherwise stated.

### Effective Dystrophin Exon Skipping Induced by Neutral PNA AOs in Aged Mdx Mice

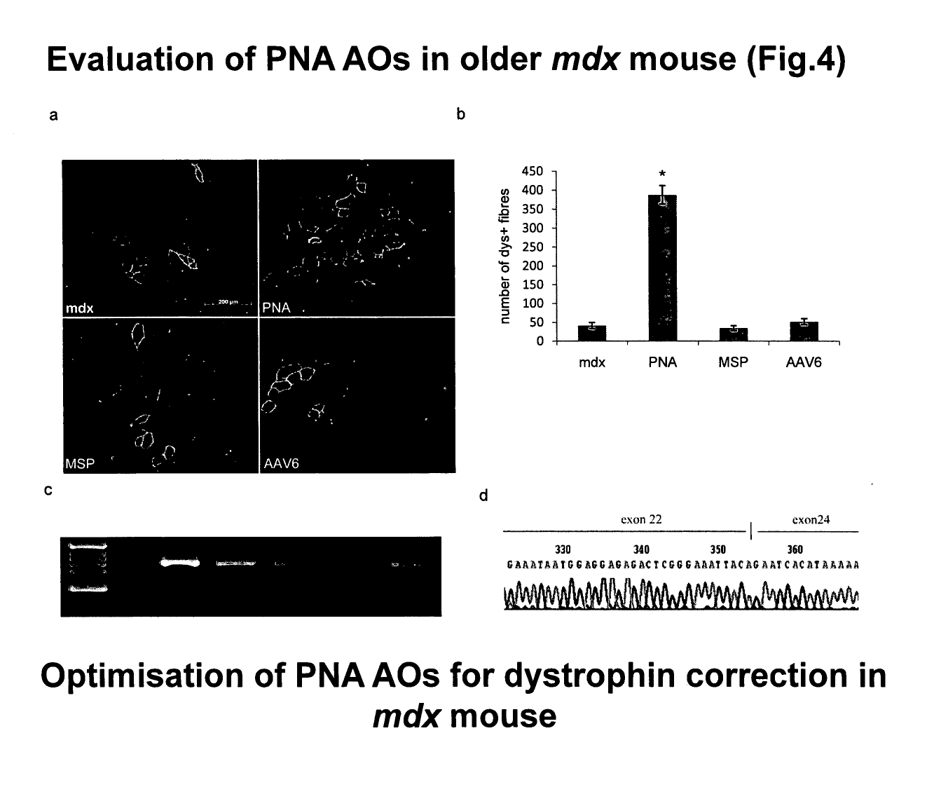

We first wished to study the potential and applicability of neutral PNA AOs and their peptide conjugate derivatives for the treatment of older DMD subjects by evaluating these compounds in aged mdx mice. 5 ug of neutral PNA AO and AAV6, MSP-conjugated PNA AOs, were studied in 12-month old mdx mice by intramuscular injection into TA muscles. Two weeks following AO injection, dystrophin-positive fibres were identified by immunohistochemical staining in muscle tissue sections. Surprisingly the number of dystrophin-postive fibres (388±24) detected in neutral PNA AO treated TA muscles was significantly higher than those treated with peptide-conjugated AOs (52±8) and compared with untreated age-matched control mdx mice (42±8) (FIG. 4a,b).

### PNA AOs Induce Long-Term Correction of Dystrophin Expression Following Intramuscular Delivery in Mdx Mice

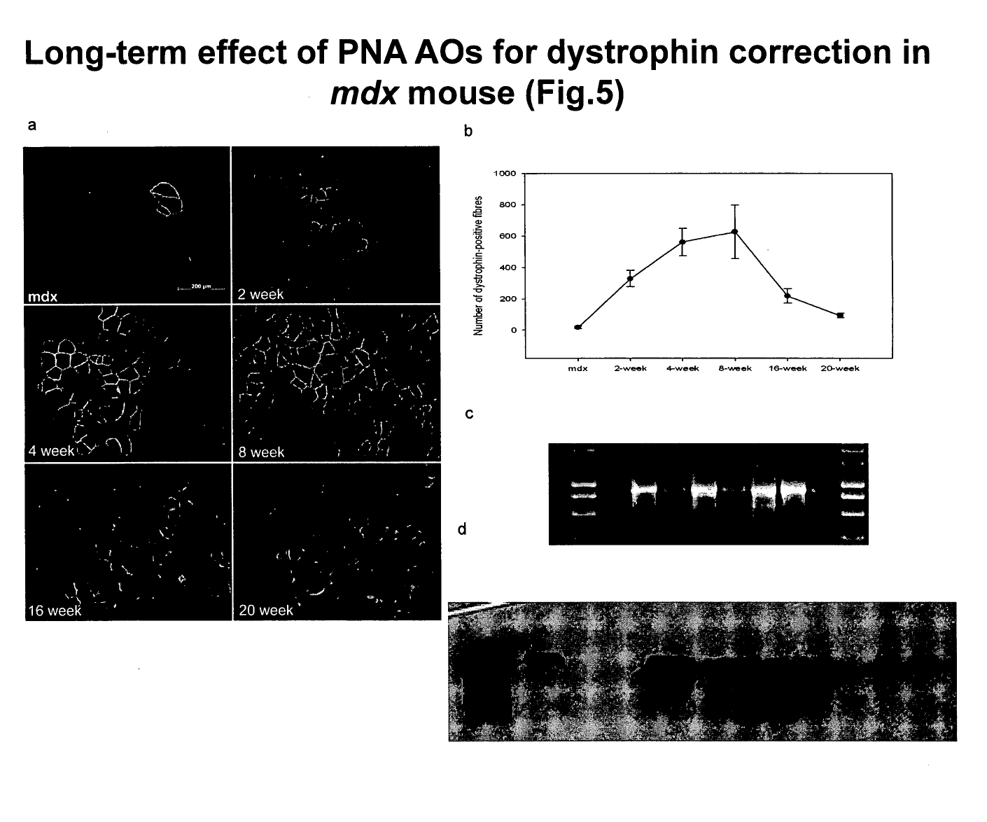

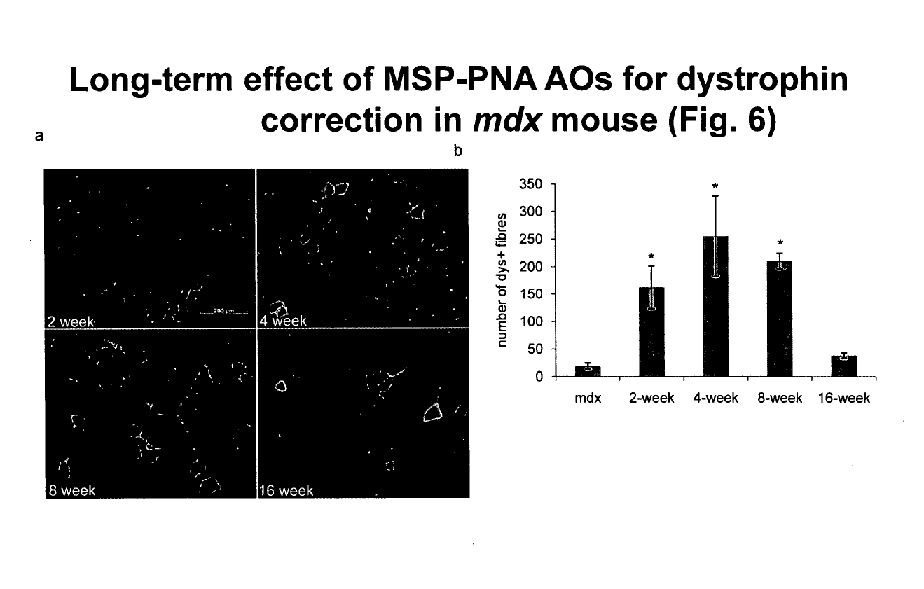

We wished to examine the duration of the therapeutic effect using PNA AOs. TA muscles of two month old mdx mice were injected with 5 ug of PNA and the efficacy of exon skipping was detected at different time points including 4, 8, 16 and 20 weeks post-injection. Treated TA muscles were harvested and dystrophin expression was identified by immunohistochemical staining. This analysis revealed greater than 650 dystrophin-positive fibres distributed uniformly in transverse muscle sections at the 8 week time point, where the numbers of dystrophin-positive fibres reached a peak (FIG. 5a). However the PNA splice correction effect was found to persist for greater than 20 weeks, although by week 16 following administration, the muscle membrane-staining had become discontinuous. Nevertheless the muscle structure and dystrophin-postive fibres were still visible at 20 weeks (FIG. 5b). The RT-PCR and Western blot results were consistent with those of the immunostaining; the RT-PCR indicated that the peak induction time for correction at the RNA level was at 4 weeks after injection where about 50% of the targeted dystrophin transcripts showed splice correction, while at the 8 week time point the level of dystrophin protein restored reached about 20% of normal levels. Western blot showed considerably reduced levels of dystrophin protein by 20 weeks post administration (FIGS. 5c and 5d).

### Systemic Evaluation of Neutral PNA AOs by Intravenous Injection

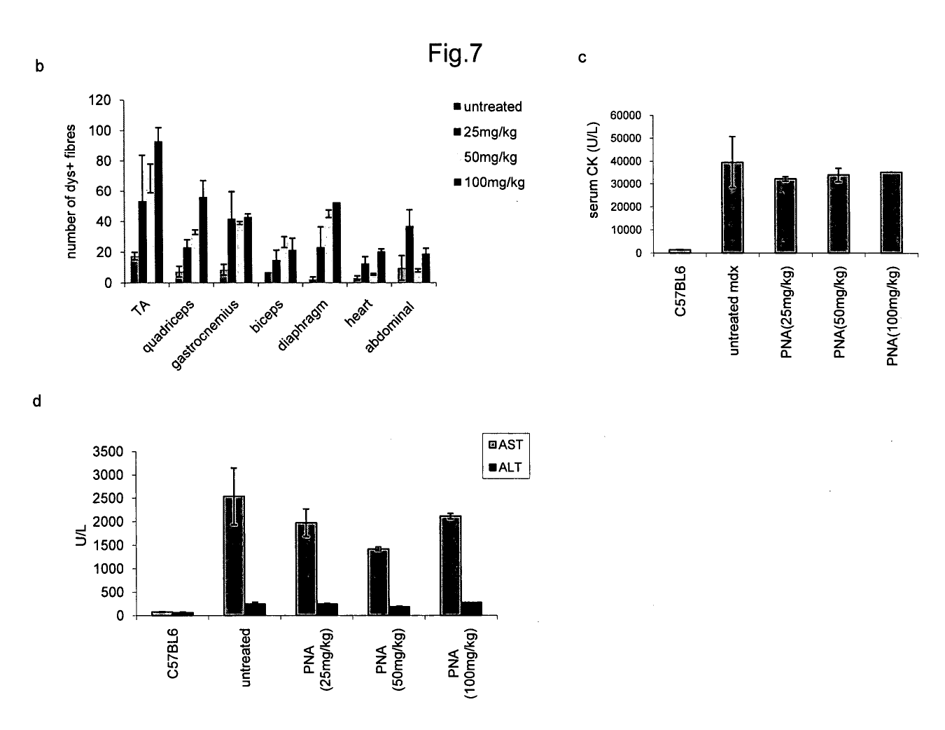

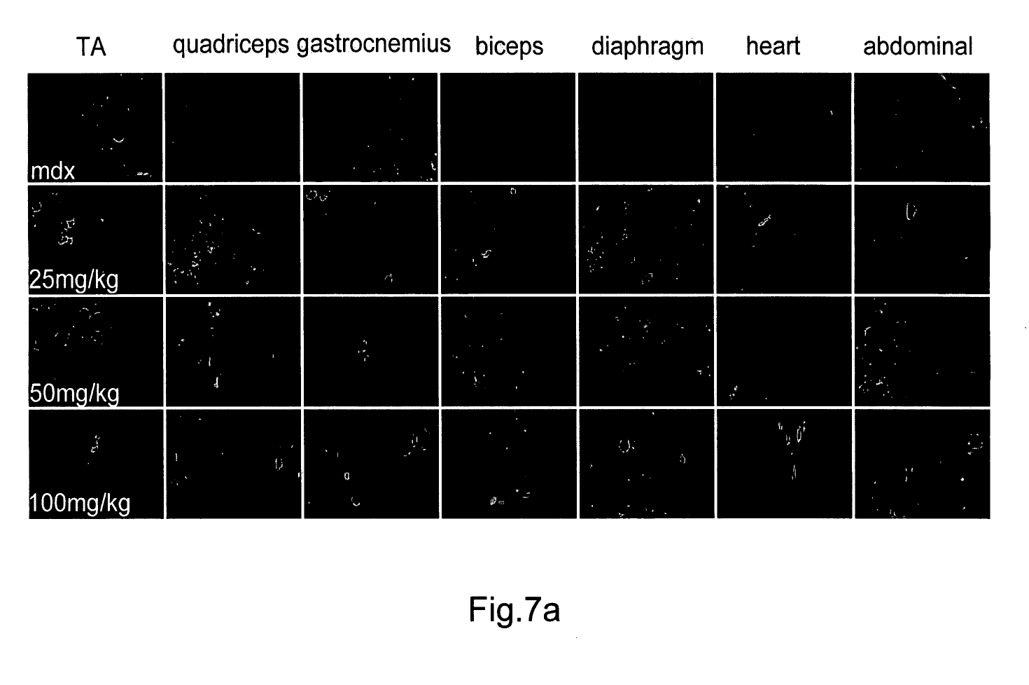

To develop AO splice correction therapy for DMD it is essential to establish effective systemic dystrophin correction since DMD is a systemic disease affecting all skeletal muscles, including the diaphragm, and cardiac muscle. Other AO chemistries, including 2′OMePS and PMO chemistries, have been reported to yield variable dystrophin splice correction following intravenous systemic delivery in mdx mice, with high systemic doses being required to detect appreciable efficacy. Here we wished to evaluate the potential of neutral PNA AOs for systemic splice correction in body-wide muscles, and initially evaluated a dose-response using three intravenous single PNA doses of 25, 50 and 100 mg/kg administered to 6 week old mdx mice via tail vein injections. Dystrophin expression was studied by immunostaining of muscle tissue sections from various peripheral muscles (including quadriceps, gastrocnemius, biceps, abdominal wall, diaphragm) and heart muscle three weeks after AO administration (FIG. 7A). The number of dystrophin positive fibres detected was low nevertheless showing a significant increase compared with untreated control mice in all muscle groups except for diaphragm and heart (FIG. 7B). No evidence of a dose response was observed over the dose range tested. RT-PCR results showed only very level of exon skipping was observed in gastrocnemius and abdominal muscles not in other muscles examined with 25 mg/kg, 50 mg/kg and 100 mg/kg (data not shown). Under detectable level of dystrophin protein was indicated by Western blot (data not shown). Measures of phenotypic correction, including serum biochemical measurements of creatine kinase and AST/ALT enzyme levels were negative (FIGS. 7C and D).

### Longer PNA AOs are More Effective for Dystrophin Splice Correction In Vivo

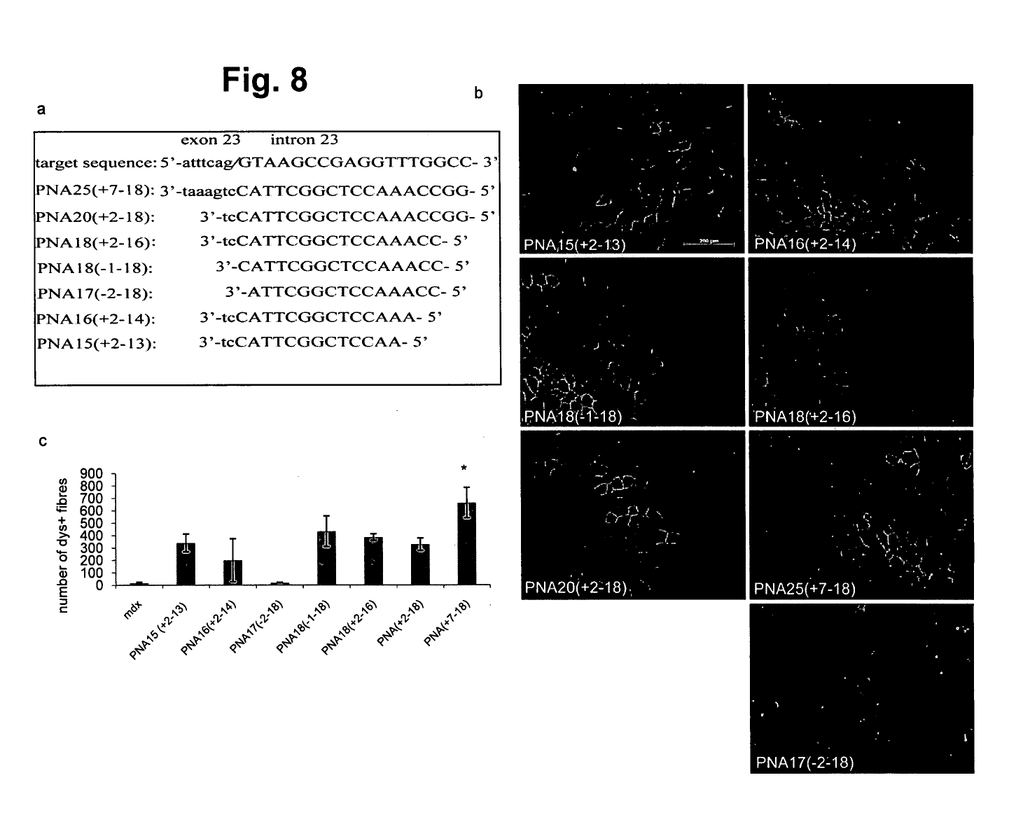

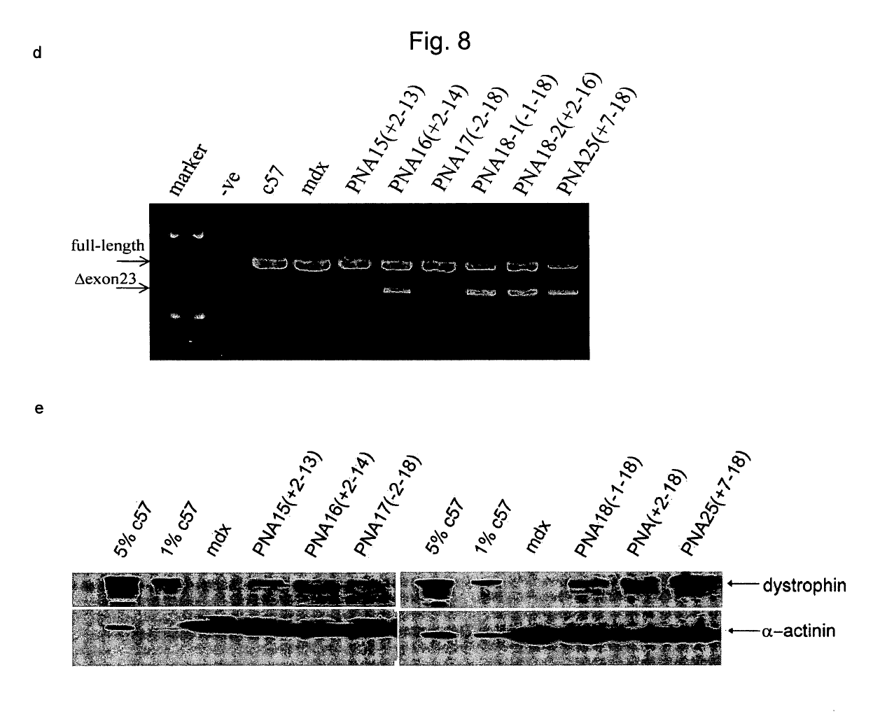

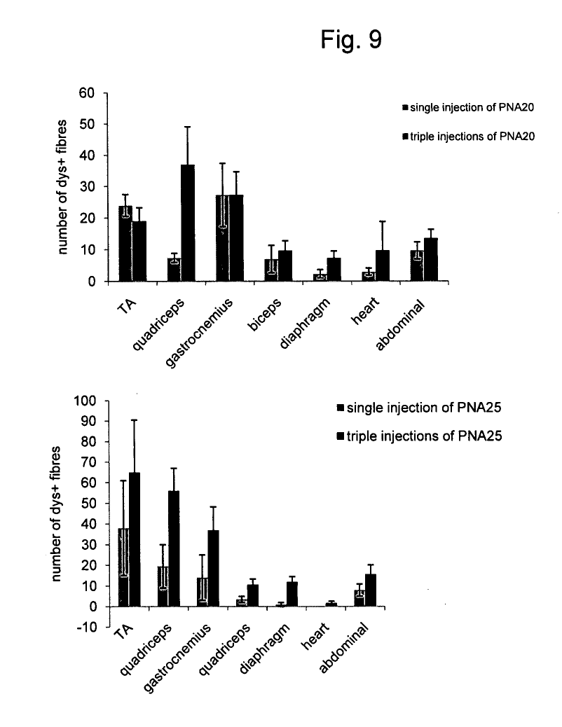

We decided to evaluate the effects of PNA length on dystrohin splice correction, initially following intramuscular injection of PNA AOs in mdx mice. Given that an earlier report studying PNA splice correction in positive read-out EGFP transgenic mice carrying an aberrant human beta-globin intron inserted in the EGFP gene had reported successful PNA splice correction using an 18 mer compound, we decided to test both shorter and longer compounds than the original 20 mer used to date in all our previous studies. PNA AOs of 15, 16, 17, 18 (two evaluated), 20 and 25 nucleotides in length (FIG. 8a) were studied. Two 18 mer compounds showed comparable activity in terms of the number of dystrophin positive fibres detected by immunochemistry (515±179) compared with the original PNA 20 mer AO, whereas an almost completely over-lapping 18 mer (shifted by two nucleotides) showed much reduced activity (387±26), confirming that AO sequence was critical factor. All AOs shorter than 18 nucleotides demonstrated reduced activity. In contrast the 25 mer PNA AO showed dramatically enhanced activity, yielding an approximately two-fold increase in the number of dystrophin positive fibres following a single intramuscular injection of 5 ug of AO (663±122) (FIGS. 8b and 8c). Approximate 50% exon skipping were detected in two 18 mer and 25 mer PNA AOs and up to 20% in the rest PNA AOs shorter than 18 mer as shown by RT-PCR (FIG. 4d). Western blot indicated the consistent efficacy as those of immunostaining and RT-PCR, up to 6% dystrophin restored with PNA25 (FIG. 8e).

### Systemic Evaluation of 25 mer PNA AOs for Exon Skipping in Mdx Mice

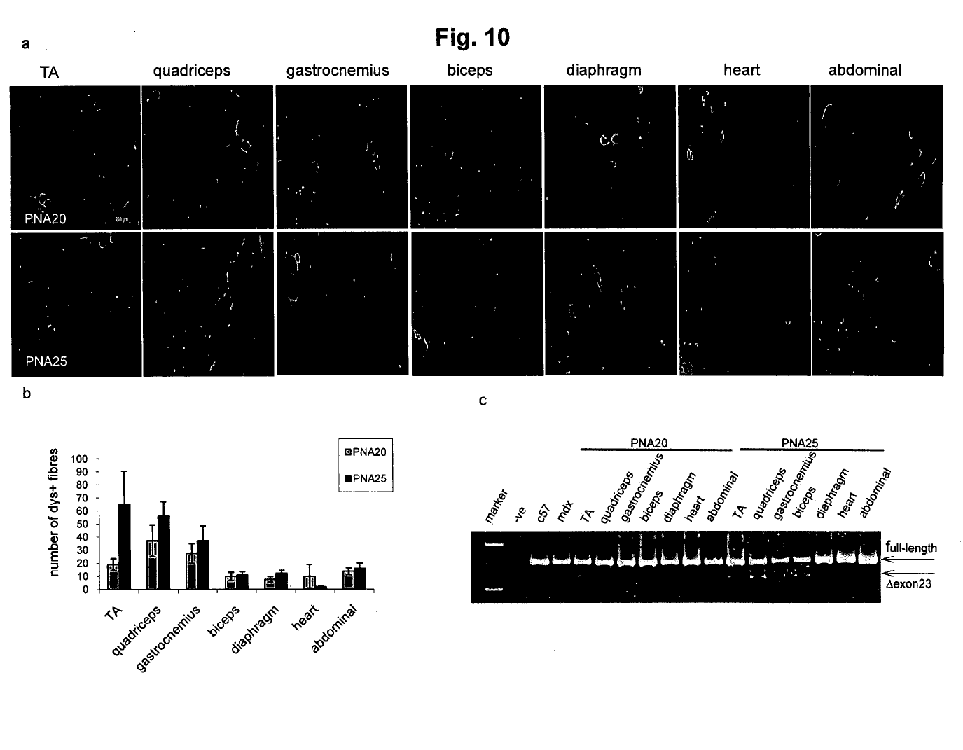

Given the two-fold improved activity of the PNA25 AOs following intramuscular administration we wished to study whether systemic administration of this longer compound would have more efficacious body-wide exon skipping activity in mdx mice. We therefore carried out tail vein injections in mdx mice using single and 3 weekly injection with 15 mg/kg doses (FIG. 10).

### PNA and PNA-Peptide Conjugates

Details of PNA and PNA-peptide conjugates are shown in Table 4. PNA and peptide-PNA conjugates were synthesized by Panagene (Korea) or Gait Lab. The PNA AO sequence against the boundary sequences of exon and intron 23 of the dystrophin gene was 5′-ggccaaaccteggettacctgaaat-3′, and designated as 20 mer PNA (M23D)—different PNA AO lengths and positions with respect to boundary region to be shown in Table 1.

### RNA Extraction and Nested RT-PCR Analysis

Total RNA was extracted from tested muscle tissues with Trizol (Invitrogen, UK) and 200 ng of RNA template was used for 20 μl RT-PCR with OneStep RT-PCR kit (Qiagen, UK). The primer sequences for the initial RT-PCR were Exon20Fo 5′-CAGAATTCTGCCAATTGCTGAG-3′ and Ex26Ro 5′-TTCTTCAGCTTGTGTCATCC-3′ for amplification of messenger RNA from exons 20 to 26. The cycle conditions were 95° C. for 30 seconds, 55° C. for 1 minute and 72° C. for 2 minutes for 25 cycles. RT-PCR product (1 μl) was then used as the template for secondary PCR performed in 250 with 0.5 U Taq DNA polymerase (Invitrogen, UK). The primer sequences for the second round were Ex20Fi 5′-CCCAGTCTACCACCCTATCAGAGC-3′ and Ex24Ri 5′-CAGCCATCCATTTCTGTAAGG-3. The cycle conditions were 95° C. for 1 minute, 57° C. for 1 minute, and 72° C. for 2 min for 25 cycles. The products were examined by electrophoresis on a 2% agarose gel.

### Intramuscular and Systemic Injection of PNA and PNA-Peptide Conjugates

For intramuscular studies the TA muscle of each experimental mdx mouse was injected with a 400 dose of PNA and PNA-peptide conjugates with saline at a final concentration of 125 μg/ml. For systemic intravenous injections, Various amount of PNA or PNA-peptide conjugates in 80 μl saline buffer were injected into tail vein of mdx mice at the final dose of 25 mg/kg, 50 mg/kg and 100 mg/kg, respectively. The animals were killed at various time points after injection by CO₂ inhalation and tissues were removed and snap-frozen in liquid nitrogen-cooled isopentane and stored at −80° C.

### Immunohistochemistry and Histology

Sections of 8 μm were cut from at least two-thirds of the muscle length of TA, quadriceps, gastrocnemius, biceps, abdominal wall and diaphragm muscles and cardiac muscle at 100 μm intervals. The sections were then examined for dystrophin expression with a polyclonal antibody 2166 against the dystrophin carboxyl-terminal region. The maximum number of dystrophin-positive fibres in one section was counted using the Zeiss AxioVision fluorescence microscope. The intervening muscle sections were collected either for RT-PCR analysis and Western blot or as serial sections for immunohistochemistry. Polyclonal antibodies were detected by goat-anti-rabbit IgGs Alexa Fluro 594 (Molecular Probe, UK). Polyclonal antibodies were detected by goat-anti-rabbit IgGs Alexa 594 (Molecular Probe, UK).

### Protein Extraction and Western Blot

The collected sections were placed in a 1.5 ml polypropylene eppendorf tube (Anachem, UK) on dry ice. The tissue sections were lysed with 150 μl protein extraction buffer containing 125 mmol/l Tris-HCl (pH=6.8), 10% sodium dodecyl sulphate, 2 mol/l urea, 20% glycerol, and 5% 2-mercaptoethanol. The mixture was boiled for 5 minutes and centrifuged. The supernatant was collected and the protein concentration was quantified by Bradford assay (Sigma, UK). Various amounts protein from normal C57BL6 mice as a positive control and corresponding amounts of protein from muscles of treated or untreated mdx mice were loaded onto sodium dodecyl sulphate polyacrylamide gel electrophoresis gels (4% stacking, 6% resolving). Samples were electrophoresed for 4 hours at 80 mA and transferred to nitrocellulose overnight at 50 V at 4° C. The membrane was then washed and blocked with 5% skimmed milk and probed overnight with DYS1 (monoclonal antibody against dystrophin R8 repeat, 1:200, NovoCastra, UK) for the detection of dytstrophin protein and α-actinin (monoclonal antibody, 1:5000, Sigma, UK) as a loading control. The bound primary antibody was detected by horseradish peroxidise-conjugated rabbit anti-mouse IgGs and the ECL Western Blotting Analysis system (Amersham Pharmacia Biosciences, UK). The intensity of the bands obtained from treated mdx muscles was measured by Image J software; the quantification is based on band intensity and area, and is compared with that from normal muscles of C57BL6 mice.

### Serum Creatinine Kinase Measurements and Other Biochemical Tests

Serum and plasma were taken from the mouse jugular vein immediately after the killing with CO₂ inhalation. Analysis of serum creatinine kinase (CK), aspartate aminotransferase (AST), alanine aminotransferase (ALT), urea and creatinine levels was performed by the clinical pathology laboratory (Mary Lyon Centre, Medical Research Council, Harwell, Oxfordshire, UK).

### Statistical Analysis

All data are reported as mean values±SEM. Statistical differences between treatment groups and control groups were evaluated by SigmaStat (Systat Software, UK) and one-tailed t test was applied.

### EXAMPLE 3

Here we report for the first time that glucose formulation facilitates the uptake, exon skipping activity and dystrophin restoration capacity of a wide range of AOs, including 2′OMePS, PNA and PMO AOs as well as PMO peptide conjugates, following intramuscular administration in mdx mice. We show that the potentiating effect of glucose is concentration-dependent, isomer specifc, can be mimicked with a range of other hexose sugars, and is potentially dependent for its effect on the activity of GLUT family of glucose transporters and active glucose metabolism (downstream metabolites). We also show that this potentiating effect extends to the systemic intravenous administration of AOs where we demonstrate enhanced exon skipping activity and correction of dystrophin expression and function in skeletal and cardiac muscles of mdx mice. We have therefore identified a clinically applicable mechanism for effectively enhancing AO delivery to muscle for dystrophin splice correction that is dependent on glucose transporter activity and that may have wider applications for nucleic acid and any other small molecule delivery to glucose-dependent tissues.

Here we report that glucose-formulated AOs demonstrate significantly improved delivery and splice correction in mdx mice. Furthermore we show that this effect is concentration-dependent, AO chemistry independent can be effected by a range of hexose sugars, requires the D-hexose isomer, and is potentially dependent on the activity of muscle glucose transporters and glucose metabolism. Moreover, such glucose-mediated AO delivery can be applied to systemic delivery protocols to enhance AO-mediated systemic splice correction in mdx mice.

### Results

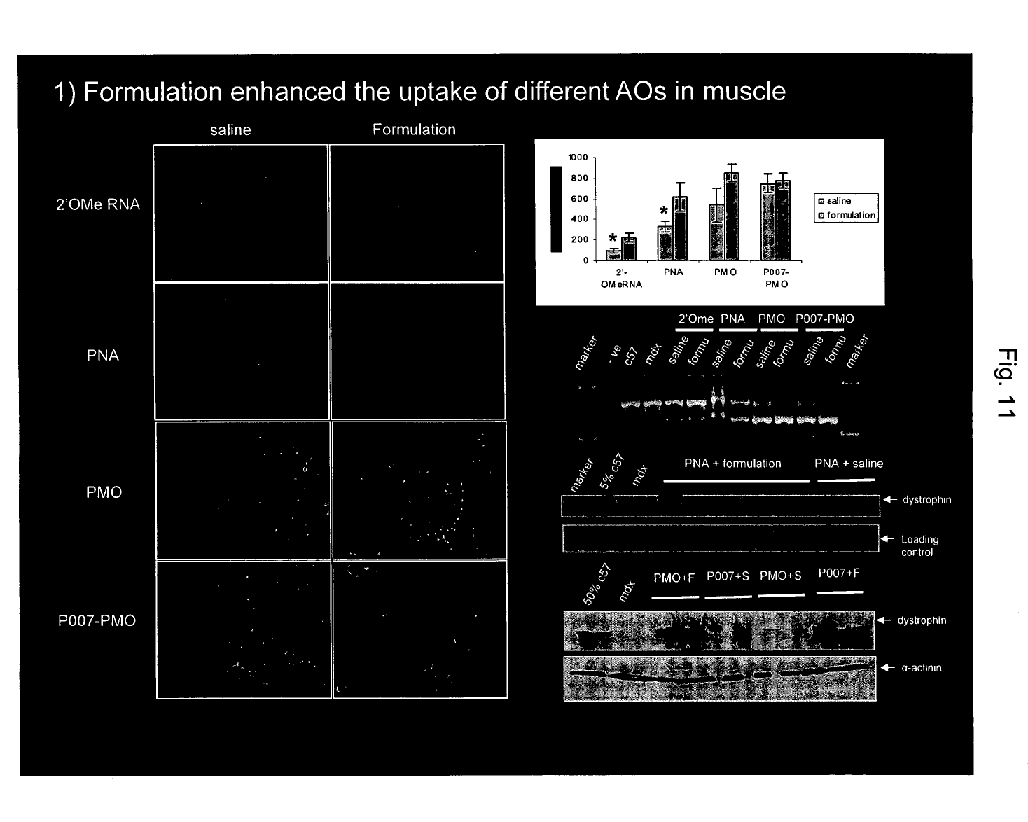

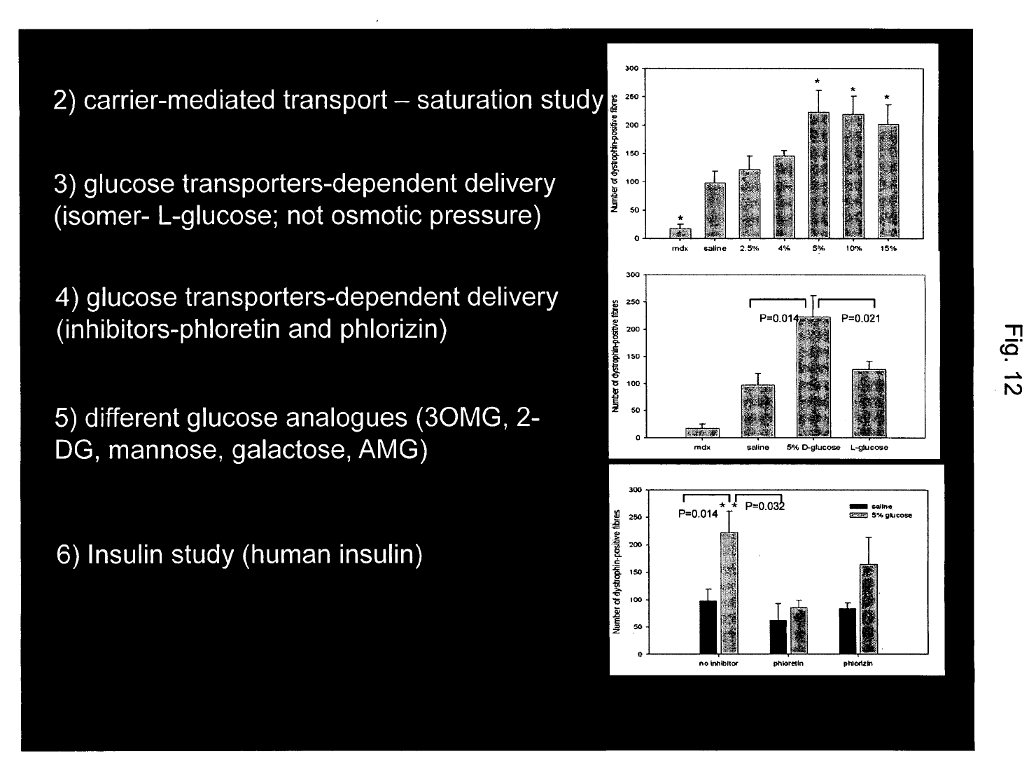

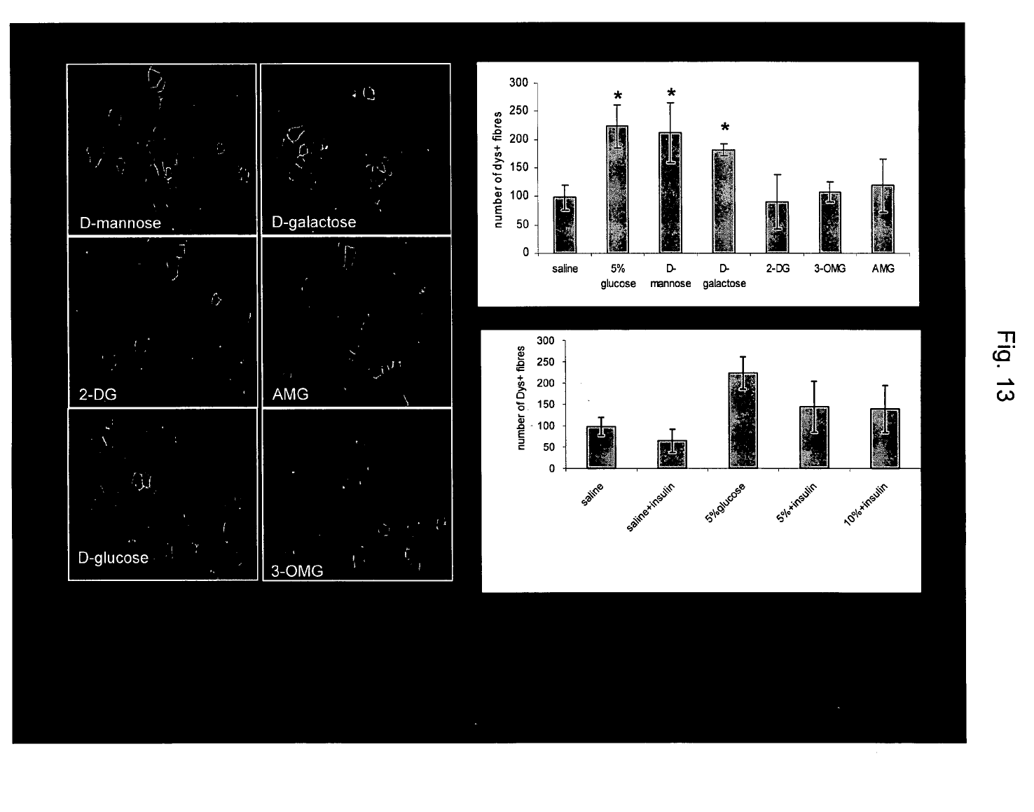

Here we study a novel delivery glucose formulation by evaluating it via local intramuscular injection into the mdx mouse tibialis anterior (TA) muscle. Initial experiments were performed in 6-8 week old mdx mice (referred to as 8 weeks) by treatment with a single dose of 5 ug of 2′OMePS AO formulated in 5% glucose. In a parallel control study, age-matched control mdx mice were treated with the same AO dose formulated in standard saline buffer. All results are shown in FIGS. 11 to 13.

### Enhanced Muscle Exon Skipping Activity of Antisense Oligonucleotides by Glucose Formulation

Two weeks after AO injection into mdx mouse TA muscle, immunohistochemistry of TA muscle tissue cryosections showed a significant increase in the number of dystrophin-positive myofibres in animals treated with the AOs formulated in 5% glucose compared with those of treated with AOs formulated in saline buffer. Given that 2′OMePS AOs are negatively charged, we wished to determine whether the glucose formulation could facilitate the uptake of other AO chemistries into muscle. We therefore tested whether the uptake and activity of uncharged peptide nucleic acid (PNA), PMO and positively-charged PMO AOs conjugated to the cell-penetrating peptide were enhanced via local intramuscular injection following glucose formulation. PNA AOs formulated with 5% glucose also showed significant improvement in dystrophin restoration compared with saline formulated compounds, however in contrast no difference was seen between glucose- and saline-formulated R9F2-PNA. Western blot and RT-PCR confirmed that in each case the expression of dystrophin protein and exon skipping activity as detected at the RNA level were consistent with the immunohistochemistry data. PMO AOs, a second neutrally charged AO, was also studied however the splice correcting activity of PMO AOs via intramuscular delivery is high in saline formulation and no significant difference in the numbers of dystrophin-positive fibres was seen with glucose-formulated PMO, however glucose-formulated PMO showed enhanced exon skipping activity at the RNA level and also increased dystrophin protein expression on Western blot.

Glucose-Enhanced AO Uptake saturates at 5-10% Glucose and is Dependent on Glucose D-Isomer Activity

To investigate whether carrier-mediated glucose uptake might be implicated in the mechanism of enhanced AO uptake into muscle, we examined the effects of a range of glucose concentrations using glucose-formulated 2′OMePS AOs. This was studied again via intramuscular administration into 8 week old mdx mice. Measuring the glucose effect in terms of the numbers of corrected dystrophin-positive myofibres, we found over range of glucose concentrations a clear dose response effect. Moreover, the glucose-enhancing effect approached saturation at glucose concentrations of between 5-10% glucose and that no further enhancement was possible with concentrations as high as 15%. These results suggested that carrier-mediated glucose transport might be implicated in the AO uptake mechanism. The activity of glucose transporters is known to be stereospecific in that only D-glucose isomers can be actively transported. To further test the idea carrier-mediated glucose uptake might be implicated in the mechanism and to rule out possible osmotic effects of the glucose formulation we investigated the activities of D- and L-glucose isomers in AO formulations. The same concentration of L-glucose was formulated with 2′OMePS AOs and delivered to 8 week old mdx mice by local intramuscular injection. Immunohistochemistry showed that the effect of glucose formulation could be abolished by substitution of the L- for the D-glucose isomer with a significant decrease in number of dystrophin-positive myofibres. This suggested that the D-glucose-mediated enhanced uptake of AOs into muscle could possibly be due to the activity of glucose transporters.

Glucose-Enhanced AO Uptake into Muscle in Mdx Mice Requires the Activity of Glucose Transporters and Glucose Metabolism

To investigate whether specific glucose transporters might be implicated directly in the mechanism of enhanced AO uptake we tested whether or not various inhibitors of glucose transporter function could abolish the enhanced uptake of 2′OMePS AOs into TA muscles in 8 week old mdx mice. Two inhibitor compounds, phloretin and phloridzin, are well-characterised inhibitors of glucose transport. Phloretin competitively inhibits carrier-mediated glucose transport in the membranes of non-epithelial cells, while phloridzin inhibits the Na+-dependent uptake of glucose at the mucosal surface of intestinal epithelial cells. Using these two compounds we found that both either partly or completed abolished the effects of the D-glucose formulation on AO uptake into muscle. The data showed that phloretin completely abolished the glucose uptake effect returning the numbers of detectable dystrophin-positive fibres to the level seen with control saline formulated AOs whereas phloridzin partially but significantly abolished the enhancing effect of the 5% glucose solution. That enhanced AO uptake and activity was dependent on a phloretin-sensitive pathway, is a hallmark of involvement of the GLUT family of facilitative glucose transporters. Given that glucose transporter-mediated glucose uptake appeared mechanistically important, we further investigated this by studying whether any products of intracellular glucose metabolism might be implicated downstream in the uptake mechanism. To do this we investigated a glucose analogue, 3-O-methyl glucose (3-OMG) that is effectively transported by glucose transporters but not subject to phosphorylation by glucokinase by using the same concentration of 3-OMG formulated with 2′OMePS AO. Two weeks following intramuscular injection TA muscles cryosections were assayed by immunohistochemistry. This showed a decrease in the number of dystrophin-positive fibres using 3-OMG formulated 2′OMePS AO compared with D-glucose formulated AO. This suggested the possibility that following glucose transport, a product(s) of glucose metabolism might be implicated in the AO uptake mechanism and enhanced AO splice correcting activity in skeletal muscle. We also tested a range of other glucose and hexose sugar analogues and found that the glucose enhancing effects could be replicated with hexose analogues also transported by glucose transporters (D-mannose and D-galactose) and that the effect was abolished by D-glucose analogues including 2-DG and AMG.

### Systemic AO Uptake and Exon Skipping Activity is Enhanced by Glucose Formulation of AO

Given the dramatic enhancement in AO uptake and exon skipping activity and dystrophin restoration observed following intramuscular delivery of glucose-formulated AO we wished to study whether or not such enhancement could also facilitate improved systemic AO delivery and splice correcting activity. Initially we chose to study whether or not systemic PMO AO delivery was enhanced following glucose formulation. We compared PMO formulated in saline with PMO formulated in either 5% glucose or formulated in glucose at a final concentration of 4.5% and delivered these via a single intravenous dose of 25 mg/kg in mdx mice. While the number of corrected dystrophin-positive fibres detectable was relatively low and variable between different muscle groups, significant enhancements were seen with both glucose formulated compounds. Evidence of enhanced dystrophin splice correction in the various muscle groups was also clearly detectable at the RNA level.

We further studied the intravenous enhancing effects by investigating the effects on a peptide conjugated PMO compound using a well characterised arginine-rich peptide (RXR)4. Glucose formulated R-PMO delivered at a single intravenous dose of 6 mg/kg showed dramatically enhanced activity compared with saline formulated R-PMO, with more effective splice correction at the RNA level and significantly improved levels of restored dystrophin protein as seen on Western blot, where protein levels in multiple peripheral muscles and heart were essentially restored to normal levels seen in wild-type C57BL TA muscle. Moreover, in accord with this data, immunohistochemistry on heart tissue cryosections showed that almost all cardiac fibres were dystrophin-positive.

### Discussion

Here we demonstrate for the first time that improved AO delivery and exon skipping activity can be obtained by formulating a range of AO chemical compounds with glucose or other hexose sugars. We find that this glucose-enhanced effect saturates at glucose concentrations of 5-10% and is directly dependent on the use of D-glucose isomers, the activity of glucose transporters and on the use of D-glucose isomers that are amenable to intracellular metabolism. This data implicates a defined cellular mechanism that is dependent on GLUT family of facilitative glucose transporters and on the effects of an as yet undefined downstream product of glucose metabolism Further, we find that such glucose formulations can also facilitate improved systemic AO delivery in mdx mice with restoration of dystophin protein expression to normal levels in multiple muscle groups including heart muscle at low systemic doses of 6 mg/kg. Therefore the use of glucose-formulated AOs has direct potential for splice correcting DMD therapy given that glucose formulation is simple and readily applicable in a clinical setting. Moreover such a mechanism of enhanced nucleic acid uptake might have mammalian biological significance/function, and might be of broader significance for the delivery of therapeutic nucleic acids to glucose-dependent tissues such as the heart and central nervous system and therefore applicable to a range of other disease types.

### AO and AO-Peptide Conjugates

Details of AO and AO-peptide conjugates are shown in Table 5. Conjugations of peptide with PMO were synthesized by a stable amide linker by Gait Lab or Panagene (Korea). The PNA AO sequence against the boundary sequences of exon and intron 23 of the dystrophin gene was 5′-ggccaaacctcggcttacctgaaat-3′, and designated as 20 mer PNA (M23D)—different PNA AO lengths and positions with respect to boundary region to be shown in Table 5.

### RNA Extraction and Nested RT-PCR Analysis

Total RNA was extracted from skeletal muscle and heart tissue with Trizol (Invitrogen, UK) and 200 ng of RNA template was used for 20 μl RT-PCR with OneStep RT-PCR kit (Qiagen, UK). The primer sequences for the initial RT-PCR were Exon20Fo 5′-CAGAATTCTGCCAATTGCTGAG-3′ and Ex26Ro 5′-TTCTTCAGCTTGTGTCATCC-3′ for amplification of messenger RNA from exons 20 to 26. The cycle conditions were 95° C. for 30 seconds, 55° C. for 1 minute and 72° C. for 2 minutes for 25 cycles. RT-PCR product (1 μl) was then used as the template for secondary PCR performed in 25 μl with 0.5 U Taq DNA polymerase (Invitrogen, UK). The primer sequences for the second round were Ex20Fi 5′-CCCAGTCTACCACCCTATCAGAGC-3′ and Ex24Ri 5′-CAGCCATCCATTTCTGTAAGG-3. The cycle conditions were 95° C. for 1 minute, 57° C. for 1 minute, and 72° C. for 2 min for 25 cycles. The products were examined by electrophoresis on a 2% agarose gel.

### Intramuscular and Systemic Injection of AO and AO Peptide Conjugates

For intramuscular studies the TA muscle of each experimental mdx mouse was injected with a 40 μl dose of PNA and PNA-peptide conjugates with saline at a final concentration of 125 μg/ml, and the contralateral muscle was injected with saline. For systemic intravenous injections, X μg PNA or PNA-peptide conjugates in 80 μl saline buffer were injected into tail vein of mdx mice at the final dose of 25 mg/kg and 6 mg/kg, respectively. The animals were killed at various time points after injection by CO₂ inhalation and tissues were removed and snap-frozen in liquid nitrogen-cooled isopentane and stored at −80° C.

### Immunohistochemistry and Histology

Sections of 8 μm were cut from at least two-thirds of the muscle length of TA, quadriceps, gastrocnemius, biceps, abdominal wall and diaphragm muscles and cardiac muscle at 100 μm intervals. The sections were then examined for dystrophin expression with a polyclonal antibody 2166 against the dystrophin carboxyl-terminal region. The maximum number of dystrophin-positive fibres in one section was counted using the Zeiss AxioVision fluorescence microscope. The intervening muscle sections were collected either for RT-PCR analysis and Western blot or as serial sections for immunohistochemistry. Polyclonal antibodies were detected by goat-anti-rabbit IgGs Alexa Fluro 594 (Molecular Probe, UK). Routine haematoxylin and eosin staining was used to examine overall muscle morphology and assess the level of infiltrating mononuclear cells. The serial sections were also stained with a panel of polyclonal and monoclonal antibodies for the detection of DAPC protein components. Rabbit polyclonal antibody to neuronal nitric oxide synthase (nNOS) and mouse monoclonal antibodies to β dystroglycan, α-sarcoglycan and β-sarcoglycan were used according to manufacturer's instructions (Novocastra, UK). Polyclonal antibodies were detected by goat-anti-rabbit IgGs Alexa 594 and the monoclonal antibodies by goat-anti-mouse IgGs Alexa 594 (Molecular Probe, UK). The M.O.M. blocking kit (Vector laboratories, Inc. Burlingame, Calif.) was applied for the immunostaining of the DAPC.

### Protein Extraction and Western Blot

The collected sections were placed in a 1.5 ml polypropylene eppendorf tube (Anachem, UK) on dry ice. The tissue sections were lysed with 150 μl protein extraction buffer containing 125 mmol/l Tris-HCl (pH=6.8), 10% sodium dodecyl sulphate, 2 mol/l urea, 20% glycerol, and 5% 2-mercaptoethanol. The mixture was boiled for 5 minutes and centrifuged. The supernatant was collected and the protein concentration was quantified by Bradford assay (Sigma, UK). Various amounts protein from normal C57BL6 mice as a positive control and corresponding amounts of protein from muscles of treated or untreated mdx mice were loaded onto sodium dodecyl sulphate polyacrylamide gel electrophoresis gels (4% stacking, 6% resolving). Samples were electrophoresed for 4 hours at 80 mA and transferred to nitrocellulose overnight at 50 V at 4° C. The membrane was then washed and blocked with 5% skimmed milk and probed overnight with DYS1 (monoclonal antibody against dystrophin R8 repeat, 1:200, NovoCastra, UK) for the detection of dytstrophin protein and α-actinin (monoclonal antibody, 1:5000, Sigma, UK) as a loading control. The bound primary antibody was detected by horseradish peroxidise-conjugated rabbit anti-mouse IgGs and the ECL Western Blotting Analysis system (Amersham Pharmacia Biosciences, UK). The intensity of the bands obtained from treated mdx muscles was measured by Image J software; the quantification is based on band intensity and area, and is compared with that from normal muscles of C57BL6 mice.

### Serum Creatinine Kinase Measurements and Other Biochemical Tests

Serum and plasma were taken from the mouse jugular vein immediately after the killing with CO₂ inhalation. Analysis of serum creatinine kinase (CK), aspartate aminotransferase (AST), alanine aminotransferase (ALT), urea and creatinine levels was performed by the clinical pathology laboratory (Mary Lyon Centre, Medical Research Council, Harwell, Oxfordshire, UK).

### Statistical Analysis

All data are reported as mean values±SEM. Statistical differences between treatment groups and control groups were evaluated by SigmaStat (Systat Software, UK) and one-tailed t test was applied.

TABLE 5
Oligonucleotide and peptide sequences

| Name | Sequence | Abbreviation | Length |
|---|---|---|---|
| M23D | 5′-GGCCAAACCTCGGCTTACCTGAAAT-3′ | PMO25 | 25 |
| | | PNA20 | 20 |
| | | 2′OMePS | 18 |
| MSP | | | |
| R9F2 | | | |
| (RXR)₄ | N-RXRRXRRXRRXRXB-C | (RXR)₄XB | 14 |

Where R = L-arginine, X = 6-aminohexanoic acid

1. A construct comprising a cell delivery peptide covalently or non-covalently attached to a biologically active compound suitable for delivery of said biologically active compound into cells, wherein the cell delivery peptide is selected from MSP, AAV6, AAV8, TAT or (RXR)₄; or a functional derivative thereof, and wherein optionally the cells are cardiac muscle, skeletal muscle, smooth muscle or contractile cells and optionally the MSP peptide is ASSLNIA, the AAV6 peptide is TVAVNLQSSSTDPATGDVHVM, the AAV8 peptide is IVADNLQQQNTAPQIGTVNSQ, TAT peptide is YGRKKRRQRRRP or the (RXR)₄ peptide is RXRRXRRXRRXR wherein R is L-arginine and X is 6-aminohexanoic acid.

2. A construct according to claim 1 wherein
the biologically active compound comprises a nucleic acid, a DNA molecule, a peptide, a protein, a DNAzyme, a Ribozyme, a chromophore, a fluorophore, and/or a pharmaceutical, and/or
the functional derivative is a polypeptide with a sequence which has homology to any of the specific sequences mentioned in claim 1 and which is able to improve delivery of the compound into cells.

3. A construct according to claim 2 wherein the nucleic acid comprises nucleic acid with phosphodiester, 2′O-methyl, 2′ methoxy-ethyl, phosphoramidate, methylphosphonate, and/or phosphorothioate backbone chemistry, peptide nucleic acid (PNA), phosphorodiamidate morpholino oligonucleotide (PMO), locked nucleic acid (LNA), glycol nucleic acid (GNA) and threose nucleic acid (TNA), plasmid DNA or small interfering RNA (siRNA).

4. A construct according to claim 2 wherein the nucleic acid comprises a sequence capable of targeting a sequence responsible for exon skipping in a mutated pre-mRNA at an exon to be skipped or included, wherein inducing exon skipping or inclusion corrects the expression of said mutated pre-mRNA and wherein without correction the mutated pre-mRNA fails to express functional protein.

5. A composition comprising the construct of claim 1 and a pharmaceutically acceptable carrier.

6. A method for delivery of a biologically active compound into a cell in vitro or in vivo comprising administering to the cell the construct of claim 1 or the composition of claim 5.

7. A method of treatment or diagnosis of the human or animal body in a subject comprising administering to the subject the construct of claim 1 or the composition of claim 5.

8. A method of treatment or diagnosis of a cardiac or skeletal muscle disease in a subject comprising administering to the subject the construct of claim 1 or the composition of claim 5.

9. The method of claim 8 wherein the skeletal muscle disease is a muscular dystrophy phenotype, optionally Duchenne muscular dystrophy (DMD).

10. The method of claim 7 wherein the construct or the composition is administered by injection, optionally by intramuscular or intravenous injection.

11. A method of delivering a biologically active compound into a cell comprising contacting said cell with a construct comprising the biologically active compound covalently linked to a peptide selected from TAT (YGRKKRRQRRRP), MSP (ASSLNIA), AAV6 (TVAVNLQSSSTDPATGDVHVM), or AAV8 (IVADNLQQQNTAPQIGTVNSQ), or a functional derivative thereof.

12. (canceled)

13. A method of delivering a nucleic acid to a cell in vivo comprising administering to the cell a composition comprising the nucleic acid and a glucose analogue wherein the nucleic acid is optionally (i) a nucleic acid as defined in claim 3, and/or (ii) in the form of a construct as defined in claim 1.

14. A method according to claim 13 for treating a cardiac or skeletal muscle disease, wherein the composition is optionally administered by intramuscular or intravenous injection and/or wherein the glucose analogue is present at a concentration of 4 to 20%.

15. The method of claim 8 wherein the construct or the composition is administered by injection, optionally by intramuscular or intravenous injection.


SEQUENCE LISTING (145 sequences)

SEQ ID NO: 1 - DNA, 20 bp, Artificial Sequence
  PNA of the invention
ggccaaacctcggcttacct

SEQ ID NO: 2 - RNA, 30 nt, Homo sapiens
ccauuuugugaauguuuucuuuugaacauc

SEQ ID NO: 3 - RNA, 21 nt, Homo sapiens
guaggucacugaagagguucu

SEQ ID NO: 4 - RNA, 30 nt, Homo sapiens
uguucagggcaugaacucuuguggauccuu

SEQ ID NO: 5 - RNA, 31 nt, Homo sapiens
ucaguuuaugauuuccaucuacgaugucagu

SEQ ID NO: 6 - RNA, 23 nt, Homo sapiens
uacgaguugauugucggacccag

SEQ ID NO: 7 - RNA, 23 nt, Homo sapiens
ugcauguuccagucguugugugg

SEQ ID NO: 8 - RNA, 29 nt, Homo sapiens
cccugugcuagacugaccgugaucugcag

SEQ ID NO: 9 - RNA, 24 nt, Homo sapiens
ucuucuguuuuuguuagccaguca

SEQ ID NO: 10 - RNA, 24 nt, Homo sapiens
cagcaguugcgugaucuccacuag

SEQ ID NO: 11 - RNA, 30 nt, Homo sapiens
guaaaagaacccagcggucuucuguccauc

SEQ ID NO: 12 - RNA, 24 nt, Homo sapiens
ucuuuaaagccaguugugugaauc

SEQ ID NO: 13 - RNA, 31 nt, Homo sapiens
cuagauccgcuuuuaaaaccuguuaaaacaa

SEQ ID NO: 14 - RNA, 30 nt, Homo sapiens
cagcuucugagcgaguaauccagcugugaa

SEQ ID NO: 15 - RNA, 31 nt, Homo sapiens
gccugagcugaucugcuggcaucuugcaguu

SEQ ID NO: 16 - RNA, 22 nt, Homo sapiens
cugcaauuccccgagucucugc

SEQ ID NO: 17 - RNA, 30 nt, Homo sapiens
cggcuaauuucagagggcgcuuucuucgac

SEQ ID NO: 18 - RNA, 23 nt, Homo sapiens
caagggcaggccauuccuccuuc

SEQ ID NO: 19 - RNA, 25 nt, Homo sapiens
uugaguucugucucaagucucgaag

SEQ ID NO: 20 - RNA, 25 nt, Homo sapiens
uuaaggccucuugugcuacaggugg

SEQ ID NO: 21 - RNA, 26 nt, Homo sapiens
cagagauuuccucagcuccgccagga

SEQ ID NO: 22 - RNA, 25 nt, Homo sapiens
uccgccaucuguuagggucugugcc

SEQ ID NO: 23 - RNA, 26 nt, Homo sapiens
uccugggcagacuggaugcucuguuc

SEQ ID NO: 24 - RNA, 25 nt, Homo sapiens
uaguuucugaaauaacauauaccug

SEQ ID NO: 25 - RNA, 30 nt, Homo sapiens
cuuguagacgcugcucaaaauuggcugguu

SEQ ID NO: 26 - RNA, 25 nt, Homo sapiens
ccgucugcuuuuucuguacaaucug

SEQ ID NO: 27 - RNA, 30 nt, Homo sapiens
ucugugauacucuucaggugcaccuucugu

SEQ ID NO: 28 - RNA, 24 nt, Homo sapiens
uucugugugaaauggcugcaaauc

SEQ ID NO: 29 - RNA, 25 nt, Homo sapiens
ugaagucuuccucuuucagauucac

SEQ ID NO: 30 - RNA, 30 nt, Homo sapiens
uuuccucucgcuuucucucaucugugauuc

SEQ ID NO: 31 - RNA, 26 nt, Homo sapiens
caagcccucagcuugccuacgcacug

SEQ ID NO: 32 - RNA, 27 nt, Homo sapiens
aucguuucuucacggacagugugcugg

SEQ ID NO: 33 - RNA, 30 nt, Homo sapiens
caggggcaacucuuccaccaguaacugaaa

SEQ ID NO: 34 - RNA, 27 nt, Homo sapiens
cugcuauuucaguuuccuggggaaaag

SEQ ID NO: 35 - RNA, 25 nt, Homo sapiens
acaucaaggaagauggcauuucuag

SEQ ID NO: 36 - RNA, 30 nt, Homo sapiens
uccaacuggggacgccucuguuccaaaucc

SEQ ID NO: 37 - RNA, 31 nt, Homo sapiens
cauucaacuguugccuccgguucugaaggug

SEQ ID NO: 38 - RNA, 27 nt, Homo sapiens
gugugaaagcugaggggacgaggcagg

SEQ ID NO: 39 - RNA, 25 nt, Homo sapiens
cgaggcuggcucaggggggaguccu

SEQ ID NO: 40 - RNA, 25 nt, Homo sapiens
ggacaggccuuuauguucgugcugc

SEQ ID NO: 41 - RNA, 27 nt, Homo sapiens
cugugcuuguguccuggggaggacuga

SEQ ID NO: 42 - RNA, 26 nt, Homo sapiens
ucucauuggcuuuccagggguauuuc

SEQ ID NO: 43 - RNA, 23 nt, Homo sapiens
caucuucugauaauuuuccuguu

SEQ ID NO: 44 - RNA, 23 nt, Homo sapiens
gucugcauccaggaacauggguc

SEQ ID NO: 45 - RNA, 30 nt, Homo sapiens
ugugaugugguccacauucuggucaaaagu

SEQ ID NO: 46 - RNA, 22 nt, Homo sapiens
cuuugagaccucaaauccuguu

SEQ ID NO: 47 - RNA, 20 nt, Homo sapiens
ggagagagcuuccuguagcu

SEQ ID NO: 48 - RNA, 24 nt, Homo sapiens
uguucagcuucuguuagccacuga

SEQ ID NO: 49 - RNA, 31 nt, Homo sapiens
caagcuuuucuuuuaguugcugcucuuuucc

SEQ ID NO: 50 - RNA, 30 nt, Homo sapiens
uucucagguaaagcucuggaaaccugaaag

SEQ ID NO: 51 - RNA, 30 nt, Homo sapiens
cuggcuuccaaaugggaccugaaaaagaac

SEQ ID NO: 52 - RNA, 30 nt, Homo sapiens
cuggcgagcaagguccuugacguggcucac

SEQ ID NO: 53 - RNA, 31 nt, Homo sapiens
gggcuucaugcagcugccugacucgguccuc

SEQ ID NO: 54 - RNA, 27 nt, Homo sapiens
cauccagucuaggaagagggccgcuuc

SEQ ID NO: 55 - RNA, 24 nt, Homo sapiens
ccucuaagacagucugcacuggca

SEQ ID NO: 56 - RNA, 24 nt, Homo sapiens
aaguugaucagaguaacgggacug

SEQ ID NO: 57 - RNA, 25 nt, Homo sapiens
gauccauugcuguuuuccauuucug

SEQ ID NO: 58 - RNA, 26 nt, Homo sapiens
ccuccuuucuggcauagaccuuccac

SEQ ID NO: 59 - RNA, 26 nt, Homo sapiens
ccaaugccauccuggaguuccuguaa

SEQ ID NO: 60 - RNA, 29 nt, Homo sapiens
ccacucagagcucagaucuucuaacuucc

SEQ ID NO: 61 - RNA, 20 nt, Homo sapiens
cuuggagucuucuaggagcc

SEQ ID NO: 62 - RNA, 25 nt, Homo sapiens
guuauccaaacgucuuuguaacagg

SEQ ID NO: 63 - RNA, 25 nt, Homo sapiens
acucaugauuacacguucuuuaguu

SEQ ID NO: 64 - RNA, 22 nt, Homo sapiens
uccucaggaggcagcucuaaau

SEQ ID NO: 65 - RNA, 27 nt, Homo sapiens
gagauggcucucucccagggacccugg

SEQ ID NO: 66 - RNA, 25 nt, Homo sapiens
ugggauggucccagcaaguuguuug

SEQ ID NO: 67 - RNA, 27 nt, Homo sapiens
gcaaagggccuucugcagucuucggag

SEQ ID NO: 68 - RNA, 27 nt, Homo sapiens
gauccucccuguucguccccuauuaug

SEQ ID NO: 69 - RNA, 26 nt, Homo sapiens
gcgcuggucacaaaauccuguugaac

SEQ ID NO: 70 - RNA, 24 nt, Homo sapiens
ugcuuuagacuccuguaccugaua

SEQ ID NO: 71 - RNA, 27 nt, Homo sapiens
gcugacugcugucggaccucuguagag

SEQ ID NO: 72 - RNA, 24 nt, Homo sapiens
gauaggugguaucaacaucuguaa

SEQ ID NO: 73 - RNA, 21 nt, Homo sapiens
caggagcuuccaaaugcugca

SEQ ID NO: 74 - RNA, 22 nt, Homo sapiens
uccucagcagaaagaagccacg

SEQ ID NO: 75 - RNA, 23 nt, Homo sapiens
ugacagccugugaaaucugugag

SEQ ID NO: 76 - RNA, 28 nt, Homo sapiens
cuggcagaauucgauccaccggcuguuc

SEQ ID NO: 77 - RNA, 22 nt, Homo sapiens
cagcaguaguugucaucugcuc

SEQ ID NO: 78 - RNA, 25 nt, Homo sapiens
cauucauuuccuuucgcaucuuacg

SEQ ID NO: 79 - RNA, 26 nt, Homo sapiens
aucucuuugucaauuccauaucugua

SEQ ID NO: 80 - RNA, 23 nt, Homo sapiens
ucugcagaauaaucccggagaag

SEQ ID NO: 81 - RNA, 25 nt, Homo sapiens
gcucaagagauccacugcaaaaaac

SEQ ID NO: 82 - RNA, 25 nt, Homo sapiens
ucugcaggauauccaugggcugguc

SEQ ID NO: 83 - RNA, 26 nt, Homo sapiens
gccauacguacguaucauaaacauuc

SEQ ID NO: 84 - RNA, 25 nt, Homo sapiens
uuuaaaaccuguuaaaacaagaaag

SEQ ID NO: 85 - RNA, 31 nt, Homo sapiens; identical to SEQ ID NO: 13

SEQ ID NO: 86 - RNA, 25 nt, Homo sapiens
cuagauccgcuuuuaaaaccuguua

SEQ ID NO: 87 - RNA, 31 nt, Homo sapiens
ucuuuucuagauccgcuuuuaaaaccuguua

SEQ ID NO: 88 - RNA, 20 nt, Homo sapiens
ccgcuuuuaaaaccuguuaa

SEQ ID NO: 89 - RNA, 25 nt, Homo sapiens
ucuuuucuagauccgcuuuuaaaac

SEQ ID NO: 90 - RNA, 25 nt, Homo sapiens
cuuuuucuuuucuagauccgcuuuu

SEQ ID NO: 91 - RNA, 25 nt, Homo sapiens
gauugcuuuuucuuuucuagauccg

SEQ ID NO: 92 - RNA, 26 nt, Homo sapiens
uggauugcuuuuucuuuucuagaucc

SEQ ID NO: 93 - RNA, 23 nt, Homo sapiens
gaucuuguuugagugaauacagu

SEQ ID NO: 94 - RNA, 23 nt, Homo sapiens
ccgucuucugggucacugacuua

SEQ ID NO: 95 - RNA, 25 nt, Homo sapiens
caugcuuccgucuucugggucacug

SEQ ID NO: 96 - RNA, 22 nt, Homo sapiens
guuauccagccaugcuuccguc

SEQ ID NO: 97 - RNA, 22 nt, Homo sapiens
guaucacuaaccugugcuguac

SEQ ID NO: 98 - RNA, 25 nt, Homo sapiens
ugauaauugguaucacuaaccugug

SEQ ID NO: 99 - RNA, 31 nt, Homo sapiens; identical to SEQ ID NO: 49

SEQ ID NO: 100 - RNA, 26 nt, Homo sapiens
accagaguaacagucugaguaggagc

SEQ ID NO: 101 - RNA, 30 nt, Homo sapiens
acaucaaggaagauggcauuucuaguuugg

SEQ ID NO: 102 - RNA, 25 nt, Homo sapiens
acaucaaggaagauggcauuucuag

SEQ ID NO: 103 - RNA, 30 nt, Homo sapiens
cuccaacaucaaggaagauggcauuucuag

SEQ ID NO: 104 - RNA, 24 nt, Homo sapiens
uucuguccaagcccgguugaaauc

SEQ ID NO: 105 - RNA, 21 nt, Homo sapiens
cacccaccaucacccucugug

SEQ ID NO: 106 - RNA, 22 nt, Homo sapiens
aucaucucguugauauccucaa

SEQ ID NO: 107 - RNA, 25 nt, Homo sapiens
aucauuuuuucucauaccuucugcu

SEQ ID NO: 108 - RNA, 23 nt, Homo sapiens
cucauaccuucugcuugaugauc

SEQ ID NO: 109 - RNA, 25 nt, Homo sapiens
gauucugaauucuuucaacuagaau

SEQ ID NO: 110 - RNA, 22 nt, Homo sapiens
auucuuucaacuagaauaaaag

SEQ ID NO: 111 - DNA, 25 bp, Homo sapiens
ctgaaggtgttcttgtacttcatcc

SEQ ID NO: 112 - RNA, 24 nt, Homo sapiens
cuguugccuccgguucugaaggug

SEQ ID NO: 113 - RNA, 31 nt, Homo sapiens; identical to SEQ ID NO: 37

SEQ ID NO: 114 - RNA, 25 nt, Homo sapiens
cauucaacuguugccuccgguucug

SEQ ID NO: 115 - RNA, 22 nt, Homo sapiens
uuggcucuggccuguccuaaga

SEQ ID NO: 116 - RNA, 25 nt, Homo sapiens
guauagggacccuccuuccaugacu

SEQ ID NO: 117 - RNA, 27 nt, Homo sapiens
gguaucuuugauacuaaccuugguuuc

SEQ ID NO: 118 - RNA, 21 nt, Homo sapiens
uacuaaccuugguuucuguga

SEQ ID NO: 119 - RNA, 25 nt, Mus musculus
ggccaaaccucggcuuaccugaaau

SEQ ID NO: 120 - RNA, 20 nt, Mus musculus
ggccaaaccucggcuuaccu

SEQ ID NO: 121 - RNA, 30 nt, Mus musculus
ggccaaaccucggcuuaccugaaauuuucg

SEQ ID NO: 122 - RNA, 30 nt, Mus musculus
uuaaaggccaaaccucggcuuaccugaaau

SEQ ID NO: 123 - RNA, 31 nt, Mus musculus
gugcuucucuguugcagaccgugccugggca

SEQ ID NO: 124 - DNA, 31 bp, Mus musculus
gcccctgauggaggcaaguuucacuuccucc

SEQ ID NO: 125 - DNA, 22 bp, Artificial Sequence
  Exon20Fo Primer
cagaattctgccaattgctgag

SEQ ID NO: 126 - DNA, 20 bp, Artificial Sequence
  Ex26Ro primer
ttcttcagcttgtgtcatcc

SEQ ID NO: 127 - DNA, 24 bp, Artificial Sequence
  Ex20Fi primer
cccagtctaccaccctatcagagc

SEQ ID NO: 128 - DNA, 20 bp, Artificial Sequence
  Ex2Ri  primer
cctgcctttaaggcttcctt

SEQ ID NO: 129 - protein, 7 aa, Artificial Sequence
  MSP peptide
ASSLNIA

SEQ ID NO: 130 - protein, 21 aa, Artificial Sequence
  AAV6 peptide
TVAVNLQSSSTDPATGDVHVM

SEQ ID NO: 131 - protein, 21 aa, Artificial Sequence
  AAV8 peptide
IVADNLQQQNTAPQIGTVNSQ

SEQ ID NO: 132 - protein, 12 aa, Artificial Sequence
  TAT peptide
YGRKKRRQRRRP

SEQ ID NO: 133 - protein, 12 aa, Artificial Sequence
  (RXR)4 peptide
RXRRXRRXRRXR

SEQ ID NO: 134 - protein, 14 aa, Artificial Sequence
  (RXR)4XB peptide
RXRRXRRXRRXRXX

SEQ ID NO: 135 - DNA, 25 bp, Artificial Sequence
  M23D
ggccaaacctcggcttacctgaaat

SEQ ID NO: 136 - DNA, 21 bp, Artificial Sequence
  Ex24Ri primer
cagccatccatttctgtaagg

SEQ ID NO: 137 - DNA, 46 bp, Artificial Sequence
  exon22-exon24 read out (Fig4)
gaaataatggaggagagactcgggaaattacagaatcacataaaaa

SEQ ID NO: 138 - DNA, 25 bp, Artificial Sequence
  target sequence
atttcaggtaagccgaggtttggcc

SEQ ID NO: 139 - DNA, 25 bp, Artificial Sequence
  PNA25
taaagtccattcggctccaaaccgg

SEQ ID NO: 140 - DNA, 20 bp, Artificial Sequence
  PNA 20
tccattcggctccaaaccgg

SEQ ID NO: 141 - DNA, 18 bp, Artificial Sequence
  PNA 18
tccattcggctccaaacc

SEQ ID NO: 142 - DNA, 16 bp, Artificial Sequence
  PNA 18 (-1-18)
cattcggctccaaacc

SEQ ID NO: 143 - DNA, 15 bp, Artificial Sequence
  PNA 17
attcggctccaaacc

SEQ ID NO: 144 - DNA, 16 bp, Artificial Sequence
  PNA 16
tccattcggctccaaa

SEQ ID NO: 145 - DNA, 15 bp, Artificial Sequence
  PNA 15
tccattcggctccaa